(19) United States
(12) Patent Application Publication
(10) Pub. No.: US 2014/0141044 A1
(43) Pub. Date: May 22, 2014

(54) Cord Colitis Syndrome Pathogen

(71) Applicants: Dana-Farber Cancer Institute, Inc., Boston, MA (US); The Broad Institute, Inc., Cambridge, MA (US); The Brigham and Women's Hospital, Inc., Boston, MA (US)

(72) Inventors: Ami S. Bhatt, Jamaica Plain, MA (US); Samuel S. Freeman, Brookline, MA (US); Chandra Sekhar Pedamallu, Belmont, MA (US); Francisco Marty, Chestnut Hill, MA (US); Matthew Meyerson, Concord, MA (US)

(21) Appl. No.: 14/078,314

(22) Filed: Nov. 12, 2013

Related U.S. Application Data
(60) Provisional application No. 61/725,281, filed on Nov. 12, 2012.

(51) Int. Cl. A61K 39/02; C12Q 1/68

(52) U.S. Cl. CPC A61K 39/0208; C12Q 1/6869
USPC 424/234.1; 435/252.1; 435/32; 514/2.4; 435/34; 435/6.11

(57) ABSTRACT

The present invention provides a novel cord colitis syndrome pathogen as well as a method for the discovery of novel viral, prokaryotic or eukaryotic genomes or genomic fragments using a sequencing-based methodology.

### RELATED APPLICATION

This application claims priority to, and the benefit of the U.S. Provisional Application No. 61/725,281 filed on Nov. 12, 2012, the contents of which are incorporated herein by reference in their entireties.

### FIELD OF THE INVENTION

The field of the invention relates to a novel cord colitis syndrome pathogen.

### INCORPORATION-BY-REFERENCE

The contents of the text file named “20363-069001US_ST25.txt”, which is created on Nov. 11, 2013 and is 48,035 KB in size, are hereby incorporated by reference in their entireties.

### BACKGROUND OF THE INVENTION

Allogeneic human stem-cell transplantation (HSCT) has become a cornerstone of therapy for patients with aggressive and refractory hematologic malignancies. While transplantation represents a potentially curative therapeutic strategy, there are significant complications associated with this form of treatment. Cytotoxic conditioning prior to administration of the stem cells and the immunological sequelae of transplantation and immunosuppression can cause significant morbidity and mortality. Conditioning and antimicrobial therapy can lead to direct toxic effects and alter the gut microbiome, thus predisposing the host to serious infections. Immunosuppression and the limited efficacy of immunologically naïve stem cells can result in life-threatening infectious complications, especially in the first year after transplantation. Despite these challenges, HSCT remains a major part of the treatment armamentarium for a variety of otherwise incurable hematologic diseases.

A major complication of transplantation is gastrointestinal toxicity, which can manifest clinically as “colitis”. Several types of colitis affect transplantation candidates, including bacteria, viral, parasitic, and immunologic (graft-versus-host disease, or GVHD). Many factors affect the likelihood of developing these different types of colitis including the conditioning regimen, immunosuppressive regimen, the extent of haplotype-matching, and stem-cell source.

Recently, a syndrome of colitis was described, which appears to be unique to umbilical cord HSCT patients. This “cord colitis syndrome” (CCS) is clinically and histopathologically distinct from other known causes of colitis in transplantation patients. Approximately 10% of patients receiving umbilical cord HSCT at a single center developed this syndrome of nonbloody, frequent stools between three and eleven months after transplantation. Histopathological evaluation of colonic biopsies revealed epithelioid granulomas without evidence of known microbial pathogens, viral cytopathic changes or signs of GVHD. A traditional infectious disease evaluation did not reveal an etiology for this syndrome.

Despite many studies and hypothesis regarding the etiology of this syndrome, the underlying pathogenesis remains unclear. Thus, there is an urgent need to identify the pathogen that causes this syndrome and an effective antibiotic agent and treatment for this syndrome.

### SUMMARY OF THE INVENTION

The present invention provides novel pathogens and methods of using these pathogens, as well as methods of identifying a novel viral, prokaryotic or eukaryotic genome or genomic fragments using a sequencing-based methodology.

The pathogens presented herein include an isolated bacterial strain that includes (i) at least one contiguous overlapping sequence (contig) selected from nucleic acid sequences of SEQ ID NOs: 1-88; (ii) at least one contig selected from nucleic acid sequences of SEQ ID NOs: 94-349; (iii) at least one open reading frame presented herein (SED ID Nos: 351-8212); (iv) a bacterial conjugation operon of SEQ ID NO: 350; (v) a bacterium of ATCC Accession No. PTA-______1; or (vi) a bacterium of ATCC Accession No. PTA-______2.

Cultures of the bacterial strains of the present invention are stored and maintained on deposit under the provisions of the Budapest Treaty with American Type Culture Collection, Manassas, Va., USA under ATCC Accession No. PTA-______1 and PTA-______2.

The present invention provides a pharmaceutical composition that includes a therapeutically effective amount of the bacterial strain presented herein.

The present invention provides a vaccine that includes a therapeutically effective amount of attenuated or inactivated bacterial strain presented herein.

The present invention provides a method of preventing, treating or alleviating a symptom of cord colitis syndrome in a subject by administering to the subject a therapeutically effective amount of a vaccine presented herein.

The present invention provides a method of screening for an antibiotic agent against the bacterial strain presented herein by contacting a living bacterium with a candidate antibiotic agent and selecting an antibiotic agent that specifically inhibits growth of the bacterium.

The present invention provides a method of screening or monitoring water supply, water source, or a water filtration system by obtaining a sample from the water supply, water source, or water filtration system and detecting the presence of the bacterial strain presented herein.

The present invention provides a method of identifying a novel viral, prokaryotic or eukaryotic genome that includes the steps of (i) collecting a nucleic acid sample from a biological sample obtained from a diseased subject; (ii) performing a genome sequencing of the nucleic acid sample and generating a mix of reads; (iii) identifying one or more unmapped reads; and (iv) assembling the one or more unmapped reads into one or more contigs, thereby identifying a novel viral, prokaryotic or eukaryotic genome. In some embodiment, the step of identifying one or more unmapped reads is carried out by taxonomic classification.

In any method presented herein, the subject may have a compromised immune system.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is a flow chart showing sample selection and experimental procedure. Formalin fixed, paraffin embedded (FFPE) samples were selected for molecular analysis based on clinical criteria. Patients for whom colon biopsies were available in the time period 120 days before and 200 days after CCS-directed antibiotic therapy were selected for inclusion in the studied cohort. DNA extraction and sequencing was followed by PathSeq analysis whereby computational subtraction was applied for the removal of human and known microbial sequences. The remaining unmapped sequencing reads and the reads with homology to known microbial sequences were then computationally assembled into longer contigs representing genomic fragments of a novel organism. Candidate pathogens, predicted by PathSeq analysis of the discovery cohort, were then detected by targeted methods such as the polymerase chain reaction in the validation cohort.

FIG. 2 is a rooted phylogenetic tree demonstrating the predicted evolutionary relationship between B. enterica and related species, which was constructed by multisequence alignment of 400 core, protein-coding genes.

FIG. 3 is a circos plot of the draft B. enterica genome assembled using unmappable reads from shotgun WGS of cord colitis samples. The whole linear genome is represented circularly in the middle track in order of descending contig size. A circular contig likely representing a plasmid was excluded from this representation. On the inner track, blue hash marks that are perpendicular to the circular genome plot indicate genes that are present in B. enterica that are not present in B. japonicum USDA 110. On the outer track, the global amino acid sequence identity of each B. enterica protein to its closest B. japonicum homolog is represented.

FIGS. 4A-4M are a serial of panels demonstrating that B. enterica is more abundant in CCS patients than in normal colon, colon cancer and GVHD controls and is present in colonic biopsies from three additional patients with CCS. The top subpanel in each figure indicates amplification of a B. enterica target after 35 cycles of PCR; the bottom subpanel indicates amplification of a human actin target after 35 cycles of PCR. A no template, negative control is also included. Results of PCR of a no template control (0), (A) five normal colon controls (p1-p5), (B) five colon cancer specimens (c1-c5), (C) three colon biopsies from patients with pathologically diagnosed GVHD (g1-3), and DNA from temporally distinct CCS biopsies from (D) patient four (samples 4a, 4b, 4c, 4e), (E) patient nine (samples 9b, 9c, 9d, 9e, 9f), (F) patient six (samples 6a, 6b) are displayed. Samples are displayed chronologically. Cord colitis syndrome-directed treatment is indicated by colored arrowheads. Microscopical images of colon tissue obtained from a patient with cord colitis are shown, including a section stained with hematoxylin and eosin (G) and a corresponding section (H-K: H lower magnitude with probe EUB; K lower magnitude with probe Brady; J: higher magnitude with probe EUB and K: higher magnitude with probe Brady), along with colon tissue from healthy controls (L and M) stained with either a universal eubacterial probe (EUB, yellow) or a bradyrhizobium-specific probe (Brady) and counterstained with 4′,6-diamidino-2-pheylindole (DAPI, orange).

FIG. 5 is a diagram showing BLASTN of contigs >2.5 kb generated by the ALLPATHS assembly of nonhuman reads of Samples 5b and 5c. Each contig is subjected to nucleotide BLAST against the NCBI nt database. The top hit was taken for each contig and the organism corresponding to the top hit is indicated on the scatter plot as described in the legend. The x-axis indicates the percentage of the contig that was contained in the top hit and the y-axis indicates the contig size.

FIG. 6 is a diagram showing GC content, size and read coverage for contigs generated by the ALLPATHS assembly of samples 5b and 5c. Each contig is indicated as a colored circle (the color corresponds to the organism encoded by the top nucleotide BLAST hit as described in FIG. 1). The size of the circle correlates with the relative size of each contig. Percent GC content is indicated on the x-axis and read coverage is indicated on the y-axis.

FIG. 7 is a histogram indicating the number of predicted B. enterica genes based on percentage global amino acid sequence identity to the closes B. japonicum homolog.

FIG. 8 is a panel of PCR results of detection of B. enterica. PCR was performed using the conditions indicated in the main text with the exception that 40 cycles of PCR were carried out. Lanes are indicated with red text and correspond to the following: 1. 100 bp MW marker; 2. CC006 (positive control)—middle scroll; 3. CC011—top scroll; 4. CC010—top scroll; 5. Non template control; 6. Hemo-D; 7. Wash 2/3 (bottle 1); 8. Wash 2/3 (bottle 2); 9. Digestion buffer; 10. Wash 1 (bottle 1); 11. Wash 1 (bottle 2); 12. Wash 1 (bottle 3); 13. Isolation additive; 14. Digestion buffer; 15. Nuclease free water.

FIG. 9 is a series of panels showing PathSeq quantification of viral reads in sequences CCS samples.

FIG. 10 is a diagram showing Phylogenetic tree (generated using PhyloPhlAn) of B. enterica and related organisms).

FIG. 11 is a diagram showing the methodological objective of “Reverse microbiology” or sequence based discovery of candidate pathogens in human and animal diseases.

FIGS. 12A-12D are diagrams showing the steps of “Reverse microbiology” approach presented herein: (A) bulk extraction of DNA (or RNA) from a complex mixture of human cells and microbial cells or particles from a diseased tissue or body fluid specimen; (B) computational subtraction of human reads followed by iterative taxonomic classification of non-human reads; (C) a computational assembly algorithm is used to generate contigs (identify areas of overlap between reads to assemble longer, contiguous read sequences); and (D) the contigs are subjected to a host of tests carried out by a classifying program (such as GAEMR—www.broadinstitute.org/software/gaemr/) in order to determine which contigs likely belong to the same organism.

### DETAILED DESCRIPTION OF THE INVENTION

The present invention is based upon, in partial, the discovery of novel bacterial species (i.e., Bradyrhizobium species) termed Bradyrhizobium enterica (B. enterica) and Bradyrhizobium enterica-like (B. enterica-like). Accordingly, the present invention provides isolated bacterial strains (e.g., Bradyrhizobium enterica, Bradyrhizobium enterica-like, and bacterial strains that includes a bacterial conjugation operon), the genomic sequence of these novel strains, compositions comprising these novel strains and methods of using these strains and the compositions. The present invention also provides methods for identifying a novel viral, prokaryotic or eukaryotic strain.

Analysis of shotgun whole genome sequencing (WGS) data from four CCS colon biopsy samples from two patients revealed over 2.5 million unclassifiable high-quality sequencing reads, suggesting the presence of a yet-unidentified microbial organism within the tissue specimens. The nonhuman reads were computationally assembled into a 7.65 Mb draft genome. Ninety-eight of 99 contiguous overlapping sequences (“contigs”) demonstrated homology to Bradyrhizobium species. The organism was named Bradyrhizobium enterica (also called B. enterica, Bradyrhizobium enterica DFCI-1 or B. enterica DFCI-1) based on the results of a rooted phylogenetic analysis. PCR confirmed the presence of B. enterica in three additional CCS patients and demonstrated absence of B. enterica in normal colon, colon cancer and graft-versus-host disease controls.

This bacterium has never been genomically described before and represents a completely novel species. The association of this bacterium with CCS suggests that B. enterica functions as an opportunistic human pathogen.

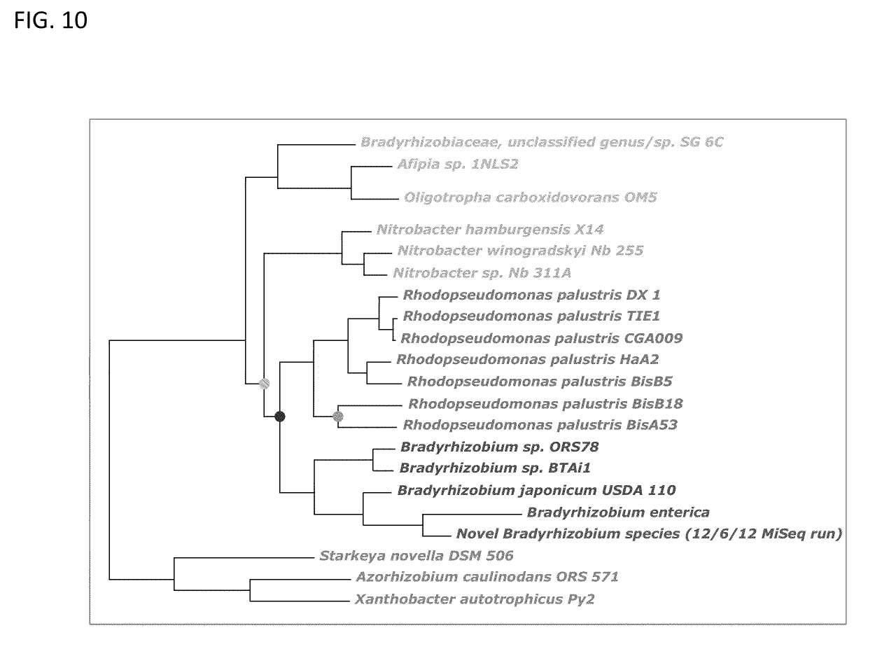

An environmental survey of patient care areas was carried out in order to establish a potential source of the infection and an organism that was similar to, but not identical to B. enterica was identified. This second novel organism (B. enterica-like or Bradyrhizobium colbertium or B. colbertium) was also determined to be in the genus Bradyrhizobium, based on a phylogenetic analysis (FIG. 10).

Both of these two bacterial species contain a conserved region that encodes a “bacterial conjugation operon” (SEQ ID NO:).

### Bradyrhizobium Strains Polynucleotide Sequences and Encoded Polypeptides

The sequences of these contigs (SEQ ID NOs: 1-88 and 94-349) are provided in the Sequence Listing as filed herein, the contents of which are hereby incorporated by reference in their entireties.

Accordingly, the present invention provides an isolated polynucleotide sequence selected from the group consisting of SEQ ID NOs: 1-88 and 94-349, or a fragment thereof. The present invention also provides an isolated polynucleotide sequence (an open reading frame, i.e., an ORF) presented herein (SED ID Nos: 351-8212). A “polynucleotide” is a nucleic acid polymer of ribonucleic acid (RNA), deoxyribonucleic acid (DNA), modified RNA or DNA, or RNA or DNA mimetics (such as PNAs), and derivatives thereof, and homologues thereof. Thus, polynucleotides include polymers composed of naturally occurring nucleobases, sugars and covalent inter-nucleoside (backbone) linkages as well as polymers having non-naturally-occurring portions that function similarly. Such modified or substituted nucleic acid polymers are well known in the art and for the purposes of the present invention, are referred to as “analogues.” Oligonucleotides are generally short polynucleotides from about 10 to up to about 160 or 200 nucleotides.

A “variant polynucleotide” or a “variant nucleic acid sequence” means a polynucleotide having at least about 60% nucleic acid sequence identity, more preferably at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% nucleic acid sequence identity and yet more preferably at least about 99% nucleic acid sequence identity with the nucleic acid sequence selected from the group consisting of SEQ ID NOs: 1-88 and 94-349.

The present invention also provides an isolated peptide or, a fragment thereof, encoded by at least one of the nucleic acid sequences of SEQ ID NOs: 1-88 and 94-349 or by at least one of the open reading frames presented herein (SED ID Nos: 351-8212). Alternatively, the present invention provides an isolated peptide selected from the group consisting of SEQ ID NOs: 8213-16021 or a fragment thereof. A fragment can be between 3-10 amino acids, 10-20 amino acids, 20-40 amino acids, 40-56 amino acids in length or even longer. Amino acid sequences having at least 70% amino acid identity, preferably at least 80% amino acid identity, more preferably at least 90% identity, and most preferably 95% identity to the fragments described herein are also included within the scope of the present invention.

As used herein, an “isolated” or “purified” nucleotide or polypeptide is substantially free of other nucleotides and polypeptides. Purified nucleotides and polypeptides are also free of cellular material or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified nucleotides and polypeptides is one that is at least 90%, 91%, 92%, 93%, 94%, 95%, 98%, 99%, or 100% (w/w) of the desired oligosaccharide by weight. Purity is measured by any appropriate standard method, for example, by column chromatography, thin layer chromatography, or high-performance liquid chromatography (HPLC) analysis. The nucleotides and polypeptides are purified and used in a number of products for consumption by humans as well as animals, such as companion animals (dogs, cats) as well as livestock (bovine, equine, ovine, caprine, or porcine animals, as well as poultry). “Purified” also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

### Recombinant Expression Vectors and Host Cells

The present invention also provides vectors, preferably expression vectors, containing at least one nucleic acid sequence of SEQ ID NOs: 1-88 and 94-349, at least one ORF presented herein (SED ID Nos: 351-8212), or derivatives, fragments, analogs or homologs thereof. As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. An exemplary vector sequence (SEQ ID NO: 89) is provided in the Sequence Listing.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. Additionally, host cells could be modulated once expressing PDX, and may either maintain or loose original characteristics.

A host cell can be any prokaryotic or eukaryotic cell. For example, any of the polypeptides or polynucleotide sequences of the present invention can be expressed in bacterial cells such as E. coli, insect cells, yeast or mammalian cells (such as Chinese hamster ovary cells (CHO) or COS cells). Alternatively, a host cell can be a premature mammalian cell, i.e., pluripotent stem cell. A host cell can also be derived from other human tissue. Other suitable host cells are known to those skilled in the art.

Vector DNA can be introduced into prokaryotic or eukaryotic cells via conventional transformation, transduction, infection or transfection techniques. As used herein, the terms “transformation” “transduction”, “infection” and “transfection” are intended to refer to a variety of art recognized techniques for introducing foreign nucleic acid (e.g., DNA) into a host cell, including calcium phosphate or calcium chloride co precipitation, DEAE dextran mediated transfection, lipofection, or electroporation. In addition transfection can be mediated by a transfection agent. By “transfection agent” is meant to include any compound that mediates incorporation of DNA in the host cell, e.g., liposome. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

Transfection may be “stable” (i.e. integration of the foreign DNA into the host genome) or “transient” (i.e., DNA is episomally expressed in the host cells).

### Antibodies Against Bradyrhizobium Strains

The present invention also includes antibodies against strains B. enterica and/or B. enterica-like, alternatively, antibodies against at least one peptide encoded by any one of the sequences of SEQ ID NOs: 1-88 and 94-349, against at least one peptide encoded by any one of the ORFs (SED ID Nos: 351-8212), against at least one peptide selected from the group consisting of SEQ ID NOs: 8213-16021 or a fragment thereof, as well as against their muteins, fused proteins, salts, functional derivatives and active fractions. The term “antibody” is meant to include polyclonal antibodies, monoclonal antibodies (MAbs), chimeric antibodies, anti-idiotypic (anti-Id) antibodies to antibodies that can be labeled in soluble or bound form, and humanized antibodies as well as fragments thereof provided by any known technique, such as, but not limited to enzymatic cleavage, peptide synthesis or recombinant techniques.

Polyclonal antibodies are heterogeneous populations of antibody molecules derived from the sera of animals immunized with an antigen. A monoclonal antibody contains a substantially homogeneous population of antibodies specific to antigens, which population contains substantially similar epitope binding sites. MAbs may be obtained by methods known to those skilled in the art. See, for example Kohler and Milstein, Nature 256:495-497 (1975); U.S. Pat. No. 4,376,110; Ausubel et al, eds., supra, Harlow and Lane, ANTIBODIES: A LABORATORY MANUAL, Cold Spring Harbor Laboratory (1988); and Colligan et al., eds., Current Protocols in Immunology, Greene Publishing Assoc. and Wiley Interscience, N.Y., (1992, 1993), the contents of which references are incorporated entirely herein by reference. Such antibodies may be of any immunoglobulin class including IgG, IgM, IgE, IgA, GILD and any subclass thereof. A hybridoma producing a MAb of the present invention may be cultivated in vitro, in situ or in vivo. Production of high titers of MAbs in vivo or in situ makes this the presently preferred method of production.

Chimeric antibodies are molecules, different portions of which are derived from different animal species, such as those having the variable region derived from a murine MAb and a human immunoglobulin constant region. Chimeric antibodies are primarily used to reduce immunogenicity in application and to increase yields in production, for example, where murine MAbs have higher yields from hybridomas but higher immunogenicity in humans, such that human/murine chimeric MAbs are used. Chimeric antibodies and methods for their production are known in the art (Cabilly et al, Proc. Natl. Acad. Sci. USA 81:3273-3277 (1984); Morrison et al., Proc. Natl. Acad. Sci. USA 81:6851-6855 (1984); Boulianne et al., Nature 312:643-646 (1984); Cabilly et al., European Patent Application 125023 (published Nov. 14, 1984); Neuberger et al., Nature 314:268-270 (1985); Taniguchi et al., European Patent Application 171496 (published Feb. 19, 1985); Morrison et al., European Patent Application 173494 (published Mar. 5, 1986); Neuberger et al., PCT Application WO 8601533, (published Mar. 13, 1986); Kudo et al., European Patent Application 184187 (published Jun. 11, 1986); Morrison et al., European Patent Application 173494 (published Mar. 5, 1986); Sahagan et al., J. Immunol. 137:1066-1074 (1986); Robinson et al., International Patent Publication, WO 9702671 (published 7 May 1987); Liu et al., Proc. Natl. Acad. Sci. USA 84:3439-3443 (1987); Sun et al., Proc. Natl. Acad. Sci. USA 84:214-218 (1987); Better et al., Science 240:1041-1043 (1988); and Harlow and Lane, ANTIBODIES: A LABORATORY MANUAL, supra. These references are entirely incorporated herein by reference.

An anti-idiotypic (anti-Id) antibody is an antibody, which recognizes unique determinants generally, associated with the antigen-binding site of an antibody. An Id antibody can be prepared by immunizing an animal of the same species and genetic type (e.g., mouse strain) as the source of the MAb with the MAb to which an anti-Id is being prepared. The immunized animal will recognize and respond to the idiotypic determinants of the immunizing antibody by producing an antibody to these idiotypic determinants (the anti-Id antibody). See, for example, U.S. Pat. No. 4,699,880, which is herein entirely incorporated by reference.

The anti-Id antibody may also be used as an “immunogen” to induce an immune response in yet another animal, producing a so-called anti-anti-Id antibody. The anti-anti-Id may be epitopically identical to the original MAb, which induced the anti-Id. Thus, by using antibodies to the idiotypic determinants of a MAb, it is possible to identify other clones expressing antibodies of identical specificity.

Accordingly, MAbs generated against any peptides of a pathogen described herein (e.g., B. enterica, B. enterica-like) and related proteins of the present invention may be used to induce anti-Id antibodies in suitable animals, such as BALB/c mice. Spleen cells from such immunized mice are used to produce anti-Id hybridomas secreting anti-Id Mabs. Further, the anti-Id Mabs can be coupled to a carrier such as keyhole limpet hemocyanin (KLH) and used to immunize additional BALB/c mice. Sera from these mice will contain anti-anti-Id antibodies that have the binding properties of the original MAb specific for a B. enterica epitope, a B. enterica-like epitope or an epitope for both strains.

The term “humanized antibody” is meant to include e.g. antibodies which were obtained by manipulating mouse antibodies through genetic engineering methods so as to be more compatible with the human body. Such humanized antibodies have reduced immunogenicity and improved pharmacokinetics in humans. They may be prepared by techniques known in the art, such as described, e.g. for humanzied anti-TNF antibodies in Molecular Immunology, Vol. 30, No. 16, pp. 1443-1453, 1993.

The term “antibody” is also meant to include both intact molecules as well as fragments thereof, such as, for example, Fab and F(ab′)₂, which are capable of binding antigen Fab and F(ab′)₂ fragments lack the Fc fragment of intact antibody, clear more rapidly from the circulation, and may have less non-specific tissue binding than an intact antibody (Wahl et al., J. Nucl. Med. 24:316-325 (1983)). It will be appreciated that Fab and F(ab′)₂ and other fragments of the antibodies useful in the present invention may be used for the detection and quantitation of an IL-18BP or a viral IL-18BP, according to the methods disclosed herein for intact antibody molecules. Such fragments are typically produced by proteolytic cleavage, using enzymes such as papain (to produce Fab fragments) or pepsin (to produce F(ab′)₂ fragments).

An antibody is said to be “capable of binding” a molecule if it is capable of specifically reacting with the molecule to thereby bind the molecule to the antibody. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

An “antigen” is a molecule or a portion of a molecule capable of being bound by an antibody which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens.

The antibodies, including fragments of antibodies, useful in the present invention may be used to detect bacteria described herein (e.g., B. enterica, B. enterica-like) quantitatively or qualitatively, or related proteins in a sample or to detect presence of cells, which express such proteins of the present invention. This can be accomplished by immunofluorescence techniques employing a fluorescently labeled antibody coupled with light microscopic, flow cytometric, or fluorometric detection.

Bradyrhizobium enterica Strain

The present invention also provides an isolated B. enterica strain comprising at least one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, or 88) contig selected from the group consisting of nucleic acid sequences of SEQ ID NOs: 1-88.

Alternatively, the present invention provides an isolated B. enterica strain of ATCC Accession No. PTA-______1. Cultures of the bacterial strains of the present invention are stored and maintained on deposit under the provisions of the Budapest Treaty with American Type Culture Collection, Manassas, Va., USA under ATCC Accession No. PTA-______1.

The present invention further provides an isolated strain that includes a bacterial conjugation operon having a nucleic acid sequence presented herein (SEQ ID NO: 350).

An “isolated” microorganism (such as an isolated B. enterica) has been substantially separated or purified away from microorganisms of different types, strains, or species. Microorganisms can be isolated by a variety of techniques, including serial dilution and culturing.

The present invention further provides a pharmaceutical composition comprising a therapeutically effective amount of inactivated or attenuated B. enterica or bacterial strain that includes a bacterial conjugation operon having a nucleic acid sequence presented herein (SEQ ID NO: 350).

Bradyrhizobium enterica-like (B. colbertium) Strain

The present invention also provides an isolated B. enterica-like strain (B. colbertium) comprising at least one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255 or 256) contig selected from the group consisting of nucleic acid sequences of SEQ ID NOs: 94-349.

The present invention also present an isolated B. enterica-like strain comprising at least one ORF presented herein (SED ID Nos: 351-8212).

Alternatively, the present invention provides an isolated B. colbertium strain of ATCC Accession No. PTA-______2. Cultures of the bacterial strains of the present invention are stored and maintained on deposit under the provisions of the Budapest Treaty with American Type Culture Collection, Manassas, Va., USA under ATCC Accession No. PTA-______2.

An “isolated” microorganism (such as an isolated B. enterica-like) has been substantially separated or purified away from microorganisms of different types, strains, or species. Microorganisms can be isolated by a variety of techniques, including serial dilution and culturing.

The present invention further provides a pharmaceutical composition comprising a therapeutically effective amount of inactivated or attenuated B. enterica-like.

### Vaccine Compositions

Also provided herein are vaccine compositions or immunogenic compositions comprising a therapeutically effective amount of inactivated or attenuated i) B. enterica; ii) B. enterica-like; iii) bacterial strains that include a bacterial conjugation having a nucleic acid sequence presented herein (SEQ ID NO: 350); or iv) any combination thereof.

A “therapeutically effective amount” of attenuated such strain(s) is an amount effective to induce an immunogenic response in the recipient. In some examples, the immunogenic response is adequate to inhibit (including prevent) or ameliorate signs or symptoms of disease (such as cord colitis syndrome), including adverse health effects or complications thereof, caused by infection with bacterial strains described herein (such as wild type B. enterica and/or B. enterica-like and/or bacterial strains having a bacterial conjugation operon). Either humoral immunity or cell-mediated immunity or both can be induced by the attenuated bacterial strains (for example in an immunogenic composition) disclosed herein. Signs and symptoms of cord colitis syndrome includes watery diarrhea.

The term “inactivation” or “inactivated” as described herein refers to treatment with inactivation agent, heat treatment, and other general methods to inactivate or kill the bacteria. The inactivation agent includes, but is not limited to, formaldehyde, binary ethyleneimine (BEI) or other suitable inactivation agents.

Attenuated bacterium refers to a bacterium having a decreased or weakened ability to produce disease (for example having reduced pathogenesis of cord colitis syndrome) while retaining the ability to stimulate an immune response like that of the natural (or wild-type) bacterium.

Attenuated vaccine refers to an immunogenic composition that includes attenuated bacteria (such as attenuated B. enterica, B. enterica-like).

Bacteria used for the vaccine may be purified prior to admixture with other formulation ingredients. The term “purified” does not require absolute purity; rather, it is intended as a relative term. Thus, for example, a purified attenuated B. enterica preparation is one in which the bacteria are more enriched than the bacteria are in its natural environment (for example within a cell). In one example, a preparation is purified such that the purified bacteria represent at least 50% of the total content of the preparation. In other examples, bacteria are purified to represent at least 90%, such as at least 95%, or even at least 98%, of all macromolecular species present in a purified preparation.

Such purified preparations can include materials in covalent association with the active agent, such as glycoside residues or materials admixed or conjugated with the active agent, which may be desired to yield a modified derivative or analog of the active agent or produce a combinatorial therapeutic formulation, conjugate, fusion protein or the like.

The present invention provides another vaccine composition. Such vaccine composition comprises at least one DNA contig selected from the group consisting of nucleic acid sequences of SEQ ID NOs: 1-88 and 94-349, at least one ORF presented herein (SED ID Nos: 351-8212) or a fragment thereof. Alternatively, the vaccine composition comprises at least one peptide encoded by a nucleic acid sequence selected from the group of SEQ ID NOs: 1-88 and 94-349 and ORFs presented herein (SED ID Nos: 351-8212). A person skilled in the art will be able to select preferred peptides, polypeptides, nucleic acid sequences or combination of thereof by testing. Usually, the most efficient peptides are then combined as a vaccine. A suitable vaccine will preferably contain between 1 and 20 peptides, more preferably 2, 3, 4, 5, 6, 7, 8, 9, 10, 11, 12, 13, 14, 15, 16, 17, 18, 19, or 20 different peptides, further preferred 6, 7, 8, 9, 10 11, 12, 13, or 14 different peptides, and most preferably 12, 13 or 14 different peptides. Alternatively, a suitable vaccine will preferably contain between 1 and 20 nucleic acid sequences, more preferably 2, 3, 4, 5, 6, 7, 8, 9, 10, 11, 12, 13, 14, 15, 16, 17, 18, 19, or 20 different nucleic acid sequences, further preferred 6, 7, 8, 9, 10 11, 12, 13, or 14 different nucleic acid sequences, and most preferably 12, 13 or 14 different nucleic acid sequences.

Any vaccine of the present invention can be a prophylactic vaccine or a therapeutic vaccine.

Any vaccine composition of the present invention may further comprise a pharmaceutical carrier, adjuvant or other co-ingredient. An adjuvant is a compound, composition, or substance that when used in combination with an immunogenic agent (such as the attenuated B. enterica bacteria disclosed herein) augments or otherwise alters or modifies a resultant immune response. In some examples, an adjuvant increases the titer of antibodies induced in a subject by the immunogenic agent. In another example, if the antigenic agent is a multivalent antigenic agent, an adjuvant alters the particular epitopic sequences that are specifically bound by antibodies induced in a subject.

Exemplary adjuvants include, but are not limited to, Freund's Incomplete Adjuvant (IFA), Freund's complete adjuvant, B30-MDP, LA-15-PH, montanide, saponin, aluminum salts such as aluminum hydroxide (Amphogel, Wyeth Laboratories, Madison, N.J.), alum, lipids, keyhole lympet protein, hemocyanin, the MF59 microemulsion, a mycobacterial antigen, vitamin E, non-ionic block polymers, muramyl dipeptides, polyanions, amphipatic substances, ISCOMs (immune stimulating complexes, such as those disclosed in European Patent EP 109942), vegetable oil, Carbopol, aluminium oxide, oil-emulsions (such as Bayol F or Marcol 52), E. coli heat-labile toxin (LT), Cholera toxin (CT), and combinations thereof.

The pharmaceutically acceptable vehicle or carrier includes, but is not limited to, solvent, emulsifier, suspending agent, decomposer, binding agent, excipient, stabilizing agent, chelating agent, diluent, gelling agent, preservative, lubricant, surfactant, adjuvant or other suitable vehicle.

### Methods of Use

The compositions of the present invention are candidates for treating or preventing certain conditions and diseases, particularly conditions and diseases associated with allogeneic human stem-cell transplantation or cancer. These compositions include: (1) an isolated polynucleotide selected from the group consisting of SEQ ID NOs: 1-88 and 94-349 and ORFs presented herein (SED ID Nos: 351-8212), or a fragment thereof; (2) an isolated peptide or, a fragment thereof, encoded by at least one of the nucleic acid sequences of SEQ ID NOs: 1-88 and 94-349 and ORFs presented herein (SED ID Nos: 351-8212); (3) an isolated pathogen (B. enterica, B. enterica-like or bacterial strain that includes a bacterial conjugation operon having a nucleic acid sequence presented herein (SEQ ID NO: 350); (4) a vector or a cell expressing at least one contig selected from the group consisting of nucleic acid sequences of SEQ ID NOs: 1-88 and 94-349 and ORFs presented herein (SED ID Nos: 351-8212); (5) a pharmaceutical composition comprising a therapeutically effective amount of one or more bacterial strains described herein or attenuated/inactivated one or more bacterial strains described herein; (6) a vaccine or an immunogenic composition comprising a therapeutically effective amount of one or more bacterial strains described herein or attenuated/inactivated one or more bacterial strains described herein.

This invention provides methods for eliciting an immune response against at least one bacterial strain described herein in a subject. The method includes administering to a subject a therapeutically effective amount of the attenuated bacteria disclosed herein (preferably in the form of an immunogenic composition or a vaccine), thereby eliciting an immune response against the bacteria in the subject.

The present invention also provides methods for treating or alleviating a symptom of conditions or disorders associated with allogeneic human stem-cell transplantation or cancer. The method includes administering to a subject, a therapeutically effective amount of a composition of the present invention.

The present invention also provides methods for preventing at least one symptom of conditions or disorders associated with allogeneic human stem-cell transplantation or cancer. The method includes administering to a subject, a therapeutically effective amount of a composition of the present invention.

The present invention further provides uses of the compositions of the present invention for the preparation of a medicament useful for the treatment of conditions or disorders associated with allogeneic human stem-cell transplantation or cancer.

The present invention further provides uses of the compositions of the present invention for the preparation of a medicament useful for the prevention of conditions or disorders associated with allogeneic human stem-cell transplantation or cancer.

As used herein, “preventing” or “prevent” describes reducing or eliminating the onset of the symptoms or complications (such as watery diarrhea) of the disease, condition or disorder associated with allogeneic human stem-cell transplantation.

One preferred disorder associated with allogeneic human stem-cell transplantation is cord colitis syndrome. Another preferred condition associated with allogeneic human stem-cell transplantation is B. enterica infection or B. enterica-like infection or an infection caused by ant pathogen described herein.

As used herein, a “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject can be male or female.

A subject can be one who had allogeneic human stem-cell transplantation or cancer. A subject can also be one who is having or will have allogeneic human stem-cell transplantation or cancer. A subject can be one who is previously infected with B. enterica or B. enterica-like or any pathogen described herein. A subject can be one who has B. enterica or B. enterica-like infection or an infection caused by any pathogen described herein. A subject can also be one who has rick of being infected with B. enterica or B. enterica-like or any pathogen described herein. A subject may have cord colitis syndrome. A subject may have comprised immune system.

A comprised immune system, also called immunodeficiency (or immune deficiency), is a state in which the immune system's ability to fight infectious disease is compromised or entirely absent. Most cases of immunodeficiency are acquired (“secondary”) but some people are born with defects in their immune system, or primary immunodeficiency. Transplant patients take medications to suppress their immune system as an anti-rejection measure. A person who has an immunodeficiency of any kind is said to be immunocompromised. An immunocompromised person may be particularly vulnerable to opportunistic infections, in addition to normal infections that could affect everyone.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 ug to 1000 ug, such as in the range from about 1 ug to 300 ug, and especially in the range from about 10 ug to 50 ug. Suitable regimens for initial administration and booster shots are also variable but are typified by an initial administration followed by subsequent inoculations or other administrations.

The manner of application may be varied widely. Any of the conventional methods for administration of a vaccine are applicable such as oral application on a solid physiologically acceptable base or in a physiologically acceptable dispersion, parenterally, by injection or the like. The dosage of the vaccine will depend on the route of administration and will vary according to the age of the person to be vaccinated and, to a lesser degree, the size of the person to be vaccinated.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and advantageously contain 10-95% of active ingredient, preferably 25-70%.

In many instances, it will be necessary to have multiple administrations of the vaccine. Especially, vaccines can be administered to prevent an infection with B. enterica or B. enterica-like, a prophylactic vaccine, and/or to treat established B. enterica or B. enterica-like infection, a therapeutic vaccine. When administered to prevent an infection, the vaccine is given prophylactically, before definitive clinical signs, diagnosis or identification of an infection is present. Prophylactic vaccines may also be designed to be used as booster vaccines. Such booster vaccines are given to individuals who have previously received a vaccination, with the intention of prolonging the period of protection. In instances where the individual has already become infected or is suspected to have become infected, the previous vaccination may have provided sufficient immunity to prevent primary disease, but as discussed previously, boosting this immune response will not help against the latent infection. In such a situation, the vaccine will necessarily have to be a therapeutic vaccine designed for efficacy against the latent stage of infection. A combination of a prophylactic vaccine and a therapeutic vaccine, which is active against both primary and latent infection, constitutes a multiphase vaccine.

The present invention also relates to a method of diagnosing any conditions or disorders associated with a bacterial strain described herein (e.g., B. enterica, B. enterica-like), such as cord colitis syndrome. The method includes steps of obtaining a sample from the subject and detecting the presence of a pathogen (e.g., bacterium) described herein (protein or DNA level). The presence of such pathogen (e.g., bacterium) indicates the subject has cord colitis syndrome or is at a risk of developing cord colitis syndrome.

By “sample” it means any biological sample derived from the subject, includes but is not limited to, cells, tissues samples, and body fluids (including, but not limited to, mucus, blood, plasma, serum, urine, saliva, and semen).

The detecting step can be carried out by any methods known in the art for determining the presence of protein or DNA of a pathogen described herein (for example, B. enterica, B. enterica-like) in the sample, such as Western Blot analysis, PCR analysis, immunohistochemistry, or any solid-phase detection methods. Exemplary agents that can be used for the detecting steps include an antibody (a monoclonal or polyclonal antibody) against B. enterica/B. enterica-like, a nucleic acid fragment and/or a polypeptide encoded by a nucleic acid fragment of B. enterica/B. enterica-like genome.

The present invention further provides a method of screening for an antibiotic agent, particularly an antibiotic agent specifically against a bacterial strain described herein (such as B. enterica, B. enterica-like). The method includes steps of contacting a living bacterium with a candidate antibiotic agent and selecting an antibiotic agent that specifically inhibits protein synthesis, cell growth cell division and/or cell viability of the tested bacterium. The phrase “an antibiotic agent specifically against a bacterial strain described herein” means the inhibitory effect of the antibiotic agent screened herein on the bacterial strain described herein is considerably greater than its inhibitory effect on other bacteria species.

The pathogen (e.g., B. enterica, B. enterica-like) is cultured in the absence or presence of the candidate antibiotic agent. At a variety of time points after treatment, protein synthesis, cell growth, cell division and/or cell viability will be assayed according to any methods available in the art, thereby screening for a pathogen selective antibiotic agent.

An antibiotic agent that prevents or disrupts protein synthesis may completely prevent protein synthesis, as defined by 98-100% loss of synthesized labeled protein as analyzed on an SDS-polyacrylamide gel or other methods available in the art. An antibiotic agent that partially inhibits protein synthesis is determined by at least, up to, and including 10%, 15%, 20%, 25%, 40%, 50%, 75%, 98% of loss of synthesized labeled protein as analyzed on an SDS-polyacrylamide gel or alternatively by an assay of uptake of labeled amino acids into a polypeptide chain that can be precipitated or trapped on a filter or other methods available in the art.

Further, an antibiotic agent that prevents or disrupts cell growth may completely prevent cell growth as defined by 98-100% retention of the same cell size without an increase in the cell size as observed by light microscopy or other methods available in the art. An antibiotic agent that partially inhibits cell growth is determined by at least, up to, and including 10%, 15%, 20%, 25%, 40%, 50%, 75%, 98% of the same cell size without an increase in the cell size.

Further, an antibiotic agent that prevents or disrupts cell division may completely prevent cell division as defined by 98-100% retention of the same cell number without an increase in cell number over time as judged by microscopy of the cells or other methods available in the art. An antibiotic agent that partially inhibits cell division is determined by at least, up to, and including 10%, 15%, 20%, 25%, 40%, 50%, 75%, 98% of the same cell number without an increase in cell number as judged by microscopy of the cells or other methods available in the art.

Still further, an antibiotic agent that prevents or disrupts cell viability may completely prevent cell viability as defined by 98-100% cell death as indicated by incorporation of Trypan Blue into the cells in a cell culture analyzed under microscope or other methods available in the art. An antibiotic agent that partially inhibits cell viability is determined by at least, up to, and including 10%, 15%, 20%, 25%, 40%, 50%, 75%, 98% of the loss of viability of the cell in a cell culture as indicated by increase of Trypan Blue stained cells or other methods available in the art.

A candidate antibiotic agent that can be tested for according to the invention include any recombinant, modified or natural nucleic acid molecule including anti-sense oligonucleotides; library of recombinant, modified or natural nucleic acid molecules; organic or inorganic compound; library of organic or inorganic compounds where the agent has the capacity to inhibit protein synthesis, cell growth, cell division and/or cell viability of B. enterica.

Test compounds for use in high-throughput screening methods may be found in large libraries of synthetic or natural substances. Numerous means are currently used for random and directed synthesis of saccharide, peptide, and nucleic acid-based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). In addition, there exist methods for generating combinatorial libraries based on peptides, oligonucleotides, and other organic compounds (Baum, C&EN, Feb. 7, 1994, page 20-26). Alternatively, libraries of natural compounds in the form of bacterial, fungal, plant and animal extracts are available from e.g. Pan Labs (Bothell, Wash.) or MycoSearch (NC), or are readily producible. Additionally, natural and synthetically produced libraries and compounds are readily modified through conventional chemical, physical, and biochemical means.

An antibiotic agent such as an antisense oligonucleotide or organic or inorganic small molecule may be administered in a eukaryotic host infected with a pathogenic agent as necessary. The antibiotic agent may be administered to, for example, a mammal, orally, cutaneously, subcutaneously, intramuscularly, intravenously, or may be inhaled as aerosols in pharmacologically suitable media daily, weekly, monthly as determined necessary in varying dosages. Administration of an antibiotic agent to, for example, a plant, may be direct spraying onto a plant or into the soil in a suitable liquid or solid medium.

Administration of, for example, small organic or inorganic molecule therapeutic agents in an individual infected with a pathogenic agent will vary depending on the potency of the small organic or inorganic molecule. For a very potent small organic or inorganic molecule inhibitor, nanogram (ng) amounts kilogram (kg) of patient, or microgram (ug) amounts per kg of patient may be sufficient. Thus, for small organic molecules, peptides, or peptoids (also called peptodimimetics), the dosage range can be for example, from about 100 ng/kg to about 500 mg/kg of patient weight, or the dosage range can be a range within this broad range, for example, about 100 ng/kg to 400 ng/kg, from about 500 ng/kg to about 1 ug/kg, from about 5 ug/kg to about 100 ug/kg, from about 150 ug/kg to about 500 ug/kg, from about 600 ug/kg to about 1 mg/kg, or from about 25 mg/kg, to about 500 mg/kg of patient weight.

The individual doses for viral gene delivery vehicles for delivery of polynucleotide inhibitors, such as antisense molecules, normally used are 107 to 109 colony forming units (c.f.u of neomycin resistance titered on HT1080 cells) per body. Dosages for, for example, adeno-associated virus (AAV) containing delivery systems are in the range of about 109 to about 1011 particles per body. Dosage of nonviral gene delivery vehicles can be 1 ug, preferably at least 5 or 10 ug, and more preferably at least 50 or 100 ug of polynucleotide, providing one or more dosages.

In all cases, routine experimentation in clinical trials will determine specific ranges for optimal therapeutic effect, for each therapeutic and each administrative protocol, and administration to specific patients will also be adjusted to within effective and safe ranges depending on the patients' condition and responsiveness to initial administrations.

All of the antibiotic agents discovered by the methods according to the present invention can be incorporated into an appropriate pharmaceutical composition that includes a pharmaceutically acceptable carrier for the agent. The pharmaceutical carrier for the agents may be the same or different for each agent. Suitable carriers may be large, slowly metabolized macromolecules such as proteins, polysaccharides, polylactic acids, polyglycolic acids, polymeric amino acids, amino acid copolymers, and inactive viruses in particles. Such carriers are well known to those of ordinary skill in the art. Pharmaceutically acceptable salts can be used therein, for example, mineral acid salts such as hydrochlorides, hydrobromides, phosphates, sulfates, and the like; an the salts of organic acids such as acetates, propionates, malonates, benzoates, and the like. A thorough discussion of pharmaceutically acceptable excipients is available in REMINGTON′S PHARMACEUTICAL SCIENCES (Mack Pub. Co., N.J. 1991). Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier. Liposomes are described in U.S. Pat. Nos. 5,422,120 and 4,762,915, WO 95/13796, WO 94/23697, WO 91/144445 and EP 524,968, and in Starrier, Biochemistry, pages 236-240 (1975) W. H. Freeman, San Francisco, Shokai, Biochem. Biophys. Acct. 600:1 (1980); Bayer, Biochem Biophys Acct 550:464 (1979); Rivet, Meth. Enzyme. 149:119 (1987); Wang, Proc. Natl. Acad. Sci. 84:785: (1987); and Plant, Anal. Biochem 176:420 (1989).

The pharmaceutically acceptable carrier or diluent may be combined with other agents to provide a composition either as a liquid solution, or as a solid form (e.g., lyophilized) which can be resuspended in a solution prior to administration. The composition can be administered by parenteral or nonparenteral routes. Parenteral routes can include local injection into an organ or space of the body or systemic injection including intravenous, intraarterial injections or other systemic routes of administration. Nonparenteral routes can include oral administration.

The present invention also provides a method for treating an infection associated with allogeneic human stem-cell transplantation, such as an infection caused by any pathogen described herein (e.g., B. enterica, B. enterica-like). The method comprises administering an antibiotic agent screened according to the method disclosed herein to a subject suspect of or infected by a pathogen (e.g., B. enterica, B. enterica-like) in an amount sufficient to reduce or prevent the infection.

Further provided by the present invention is a method of screening or monitoring water supply, water source, or a water filtration system. The method comprises steps of obtaining a sample from the water supply, water source, or water filtration system and detecting the presence of i) bacterial conjugation operon (SEQ ID NO: 350) or a fragment thereof; or ii) protein or DNA or a fragment thereof of B. enterica and/or B. enterica-like. Preferably, the water supply, water source or water filtration system screened and/or monitored herein is located in a hospital. More preferably, the water supply, water source or water filtration system screened and/or monitored herein is used for a subject who has a comprised immune system.

Any methods available in the art that are suitable for detecting i) bacterial conjugation operon (SEQ ID NO: 350) or a fragment thereof; or ii) protein or DNA or a fragment thereof of B. enterica and/or B. enterica-like can be used. For example, it can be detected by an antibody (monoclonal or polyclonal) against B. enterica/B. enterica-like. Alternatively, it can be detected by an isolated oligonucleotide that is specific to bacterial conjugation operon (or a fragment thereof) or genome DNA (or a fragment thereof) of B. enterica/B. enterica-like. The oligonucleotide probes may be at least 15 nucleotides in length. In alternate embodiments, oligonucleotide probes may range from about 20 to 200, or from 40 to 100, or from 45 to 80 nucleotides in length.

DNA isolated from the water supply, water source, or water filtration system can be amplified, e.g., using PCR. Alternatively, it can be detected by PCR using primers specific for bacterial conjugation operon (or a fragment thereof) and/or genome DNA (or a fragment thereof) of B. enterica/B. enterica-like.

Also provided herein are methods for water purification and/or decontamination. The methods include steps of obtaining a sample from a water supply, water source, or water filtration system; detecting the presence of i) bacterial conjugation operon or a fragment thereof; or ii) protein or DNA or a fragment thereof of B. enterica and/or B. enterica-like in the water supply, water source, or water filtration system; and purifying/decontaminating the water supply, water source, or water filtration system when i) bacterial conjugation operon or a fragment thereof; or ii) protein or DNA or a fragment thereof of B. enterica and/or B. enterica-like is present. Water purification and/or decontamination can be carried out by any methods known in the art, for example, by chemical agents, radiation chambers, electrostatic treatment, and filters.

The present invention further provides a method of identifying a novel viral, prokaryotic or eukaryotic genome that includes steps of (i) collecting/providing a nucleic acid sample from a biological sample obtained from a diseased subject; (ii) performing a genome or RNA sequencing of the nucleic acid sample and generating a mix of reads; (iii) identifying one or more unmapped reads; and (iv) assembling the one or more unmapped reads into one or more contigs, thereby identifying a novel viral, prokaryotic or eukaryotic genome. Any methods known in the art can be used to identify one or more unmapped reads, for example utilizing taxonomic classification. A biological sample can be any tissue, body fluid, body secretion, or body excretion from the diseased subject. For example, the subject is suffering from a post-HSCT colitis syndrome. For example, the subject is undergoing cancer treatment. For example, the subject is suffering from a pathogen infection.

Current microbiological methods used for diagnosis of human diseases in the clinical setting are biased to the identification of known organisms (with known growth, morphological, behavioral or sequence-based characteristics). Thus, the existing methods used bias against the discovery of unknown or unanticipated microorganisms. The method described herein circumvents this inherent bias.

In certain illustrative embodiments, the method includes the following steps.

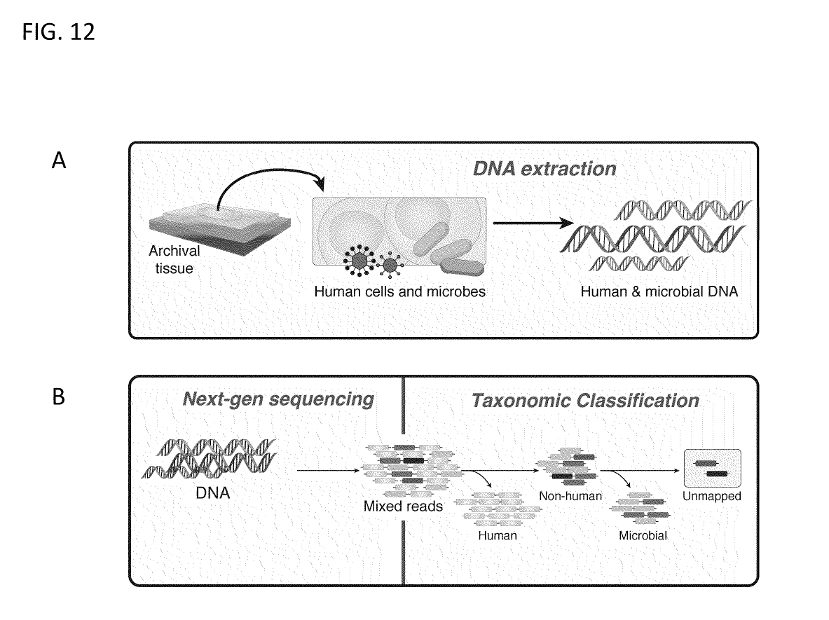

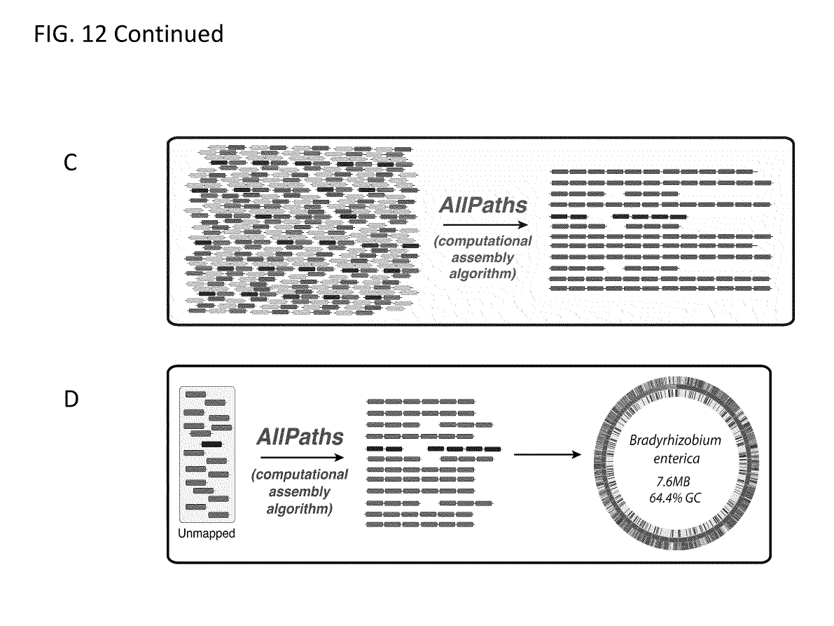

The first step is to obtain diseased human or animal tissue or body fluid (or body secretion or excretion). Total DNA or RNA can be extracted from the sample (which is theorized to be a mixture of human and non-human microbial particles or cells as demonstrated in FIG. 12A.

The resultant DNA (or RNA) is subjected to next generation sequencing, which generates a mixed population of reads from human and other sources. These sequences may be quality filtered and are then taken forward for taxonomic classification using a homology based classifier or alignment system (one possible approach is to use a program such as PathSeq (Kostic et al, Nature Biotechnology, 2011)). Known microbial reads are assigned to a taxonomic classifier and the resultant data can be used for the identification of rare or abundant microorganisms that may be candidate pathogens. In most cases, a subset of reads will remain unclassifiable or “unmapped” (as outlined in FIG. 12B).

The remaining unmapped reads (or all nonhuman reads) can be taken forward for the generation of longer “contigs” or contiguous sequences that are generated by identifying regions of overlap between reads. This can be performed using computational methods that rely on “overlap consensus method”, de Bruijn graph theory based methods, “greedy extension methods”, or other computational methods. For the work described herein, de Bruijn graph based assemblers in the programs VELVET and ALLPATHS was used. This resulted in the generation of longer sequences that are thought to comprise regions of the novel or divergent organism's genome (FIG. 12C).

Finally, the contigs are subjected to a host of tests carried out by a classifying program (such as GAEMR—www.broadinstitute.org/software/gaemr/) in order to determine which contigs likely belong to the same organism (as more than one organism without an existing draft genome may exist within the sample set) (FIG. 12D).

### Kits

A composition of the present invention may, if desired, be presented in a kit (e.g., a pack or dispenser device) which may contain one or more unit dosage forms containing the composition, for example (1) an isolated polynucleotide selected from the group consisting of SEQ ID NOs: 1-88 and 94-349 and ORFs presented herein (SED ID Nos: 351-8212), or a fragment thereof; (2) an isolated peptide or, a fragment thereof, encoded by at least one of the nucleic acid sequences of SEQ ID NOs: 1-88 and 94-349 and ORFs presented herein (SED ID Nos: 351-8212); (3) an isolated pathogen (B. enterica, B. enterica-like or bacterial strain that includes a bacterial conjugation operon having a nucleic acid sequence presented herein); (4) a vector or a cell expressing at least one contig selected from the group consisting of nucleic acid sequences of SEQ ID NOs: 1-88 and 94-349 and ORFs presented herein (SED ID Nos: 351-8212); (5) a pharmaceutical composition comprising a therapeutically effective amount of one or more bacterial strains described herein or attenuated/inactivated one or more bacterial strains described herein; (6) a vaccine or an immunogenic composition comprising a therapeutically effective amount of one or more bacterial strains described herein or attenuated/inactivated one or more bacterial strains described herein.

The pack may for example comprise metal or plastic foil, such as a blister pack. The pack or dispenser device may be accompanied by instructions for administration. Compositions comprising a composition of the invention formulated in a compatible pharmaceutical carrier may also be prepared, placed in an appropriate container, and labeled for treatment of an indicated condition. Instructions for use may also be provided.

The kits may also include a plurality of detection reagents that detect the presence of a pathogen described herein. For example, the kit includes antibodies or fragments thereof, polypeptide, aptamers or oligonucleotide sequences. The kit may contain in separate containers an aptamer or an antibody, control formulations (positive and/or negative), and/or a detectable label such as fluorescein, green fluorescent protein, rhodamine, cyanine dyes, Alexa dyes, luciferase, radiolabels, among others.

Instructions (e.g., written, tape, VCR, CD-ROM, etc.) for carrying out the assay may be included in the kit. The assay may for example be in the form of PCR, Western Blot analysis, Immunohistochemistry (IHC), immunofluorescence (IF), sequencing and Mass spectrometry (MS) as known in the art.

### EXAMPLES

### Example 1

### Methods

Sample Selection, DNA Extraction and Preparation and Sequencing of Bar-Coded Libraries

The 11 patients that comprised the original CCS cohort were chosen for further investigation. A retrospective clinical chart review was performed and identified gastrointestinal biopsies for these 11 patients. During further review of the gastrointestinal biopsies from the 11 patients of the original CCS cohort, we noted that five patients had undergone lower gastrointestinal endoscopy with biopsy both before and after antibiotic treatment initiation for CCS, and 16 of these colonic biopsies were selected for further investigation (Table 1a). FFPE preserved control tissues were obtained from: histologically normal mucosa from five healthy patients who had undergone screening colonoscopy; three umbilical cord blood stem-cell transplantation patients with pathologically confirmed intestinal GVHD; and DNA from five colon cancer resection specimens, which were previously described. Institutional review board approval was granted for this study and all patient samples were de-identified.

After the first 20 μm of each FFPE block was removed, two 20 μm shaves were then obtained and taken forward for DNA extraction (RecoverAll total nucleic acid isolation, AM1975, Ambion, Grand Island, N.Y., USA). Samples for which >25 ng DNA was extracted were taken forward for sequencing; samples for which <25 ng DNA was extracted were reserved for validation studies. Bar-coded libraries were prepared from two temporally separated samples from each of two CCS patients as described. 18 Paired-end 76-bp or 101-bp sequencing was performed at a different sequencing center (using an Illumina V3 HiSeq platform) for each patient in order to control for possible contamination.

Computational Subtraction Followed by De Novo Assembly of Unmappable Reads

Quality filtering of all sequencing reads was performed followed by sequential computational subtraction of human reads, known microbial, and viral reads using the PathSeq software (version 1.2; www.broadinstitute.org/software/pathseq/) as previously described.

Non-human reads from samples 11b and 11d were pooled and subjected to de novo assembly using two different assembly methods: the (1) VELVET and (2) ALLPATHS software packages. Contigs that comprised the novel genome were aligned to the NCBI nt database using the Basic Local Alignment Search Tool for Nucleotide sequences (BLASTN). Contigs that had homology to Bradyrhizobia or related genera, had similar sequencing coverage, and had similar % GC content to the mean GC content were included in the deposited draft genome. A subset of contigs was linked to one another using paired reads to generate supercontigs.

Comparative Genomic Analysis

The supercontigs generated by the de novo assembly comprised the draft genome of a novel organism, termed Bradyrhizobium enterica. This draft genome will be deposited (NCBI Bioproject PRJNA174084, accession number AMFB00000000; strain name B. enterica DFCI-1) and was annotated using the Prodigal automated annotation tool, as described. Rooted phylogenetic analysis was performed using a subset of 400 core genes as described (huttenhower.org/phylophlan, manuscript submitted). Bootstrap analysis was carried out.

Comparative genomic analysis was performed. Global amino acid sequence alignment was performed using the Needleman-Wunsch algorithm and percentage identity between each B. enterica gene and its closest homolog in B. japonicum, was determined.

Polymerase Chain Reaction (PCR) Amplification of a B. enterica Target and Human Actin Control

Primers for PCR were designed and generated against a nonconserved region of the provisional B. enterica genome using the PrimerQuest program (Integrated DNA Technologies, Coralville, Iowa, USA). These primers (Forward primer 5′-TCGAGGGCTACGGCTTGAAGATTT-3′ (SEQ ID NO: 90), Reverse primer 5′ ACAACGTGTTGCCGCCAATATGAG-3′(SEQ ID NO: 91)) amplify a 367 bp target, which spans an intergenic region (supercontig 17, by 152,156-152,522). Primers that target the human actin gene (Forward primer 5′-GCGAGAAGATGACCCAGATC-3′(SEQ ID NO: 92), Reverse primer 5′-CCAGTGGTACGGCCAGAGG-3′(SEQ ID NO: 93)) amplify a 102 bp target.

### Example 2

### Results and Conclusions

Shotgun Sequencing and PathSeq Analysis of Colon Biopsies from Patients with CCS

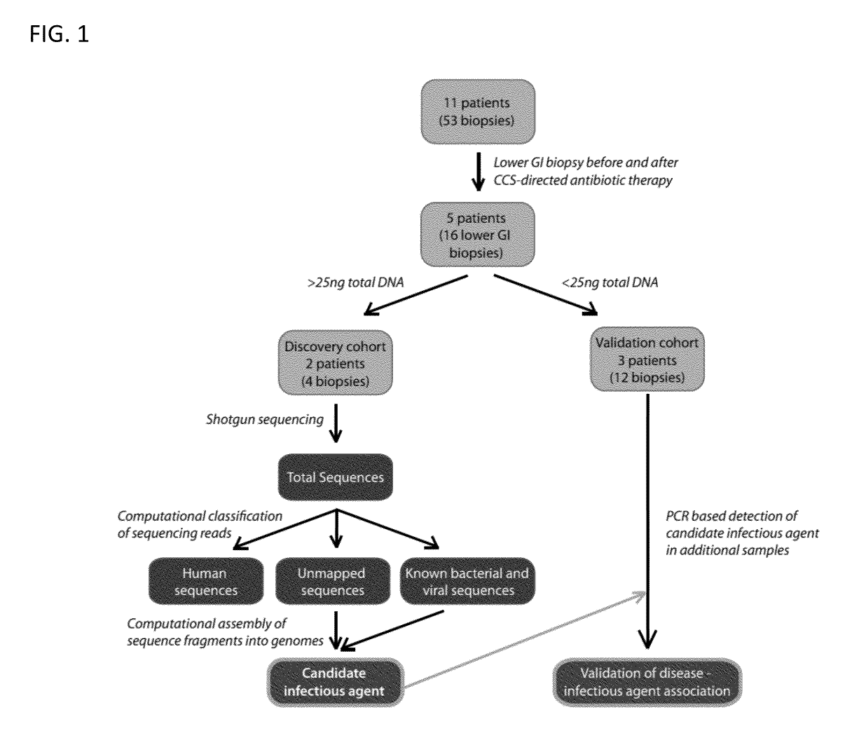

Of all biopsies performed for the 11 patients with cord colitis, those obtained within 120 days before or 200 days after antibiotic therapy were selected for further analysis (FIG. 1, Table 1a). DNA was extracted and two temporally separated colonic biopsy specimens from each of two affected patients (samples 5b, 5c, 11b and 11d; Table 1a), chosen due to DNA yield >25 ng from the extraction step, were taken forward for massively parallel sequencing. Bar-coded sequencing libraries were prepared and subjected to Illumina V3 sequencing as described. Sequential computational subtraction of human reads, known microbial reads, and viral reads was performed as described (Table 1b). Over 2.5 million reads remained unmapped suggesting the presence of abundant sequences absent from the bacterial reference database used (Table 1b).

Genome Assembly and Comparative Genomics

A pooled sets of reads from samples 11b and 11d were subjected to de novo assembly using both the VELVET and ALLPATHS software packages. ALLPATHS generated the largest number of total contigs >2.5 kb. Ninety-nine contigs generated by this method were assembled into 89 supercontigs/scaffolds and were manually reviewed; one supercontig (3,621 bp) was removed, as it exhibited high sequence similarity to a SEN virus. Another supercontig was found to encode a 126 kb circular plasmid (contig000032, scaffold00025) with high homology to a plasmid element from Bradyrhizobium BTAi (accession number CP000495.1), but is absent in B. japonicum. The 88 remaining supercontigs all contained regions of high homology to B. japonicum (which comprises a single circular chromosome of 9,105,828 bp) and 86 of the 88 supercontigs had a GC content between 60 and 66%. The final draft genome size (including the plasmid) was 7,645,871 with 64.4% GC content. Given the high GC content of the organism and the single fragment-length library method used for sequencing, small areas of the genome are likely to remain unassembled. However, the over 35-fold coverage of the genome suggests that the majority of the genome has been discovered. Seventy one hundred and twelve open protein-encoding genes were predicted within the provisional genome using the Prodigal genome annotation tool.

Phylogenetic Analysis was Performed Using the PhyloPhlan Software

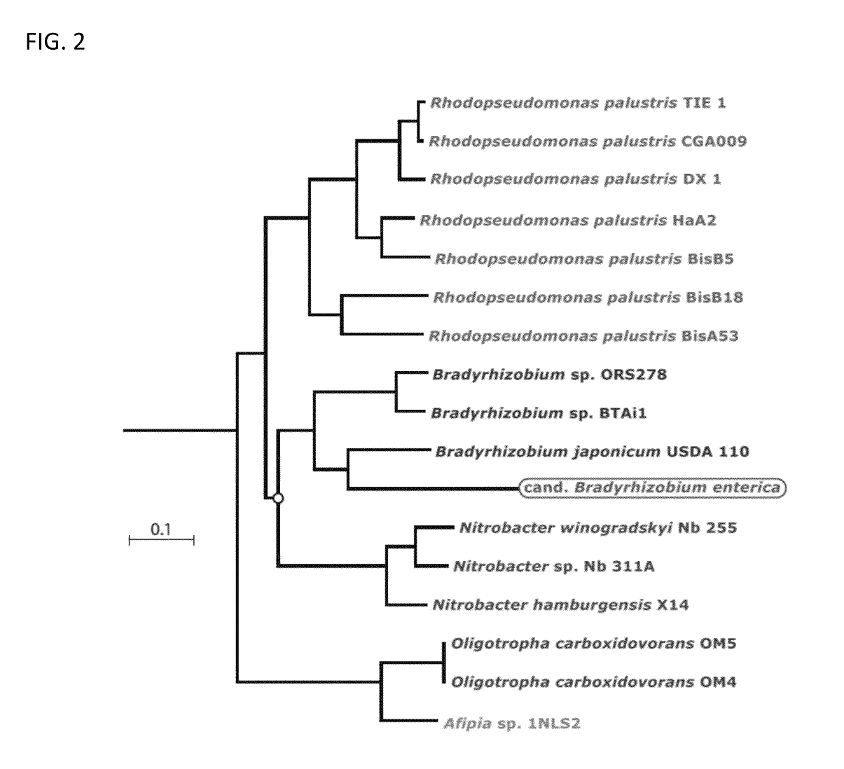

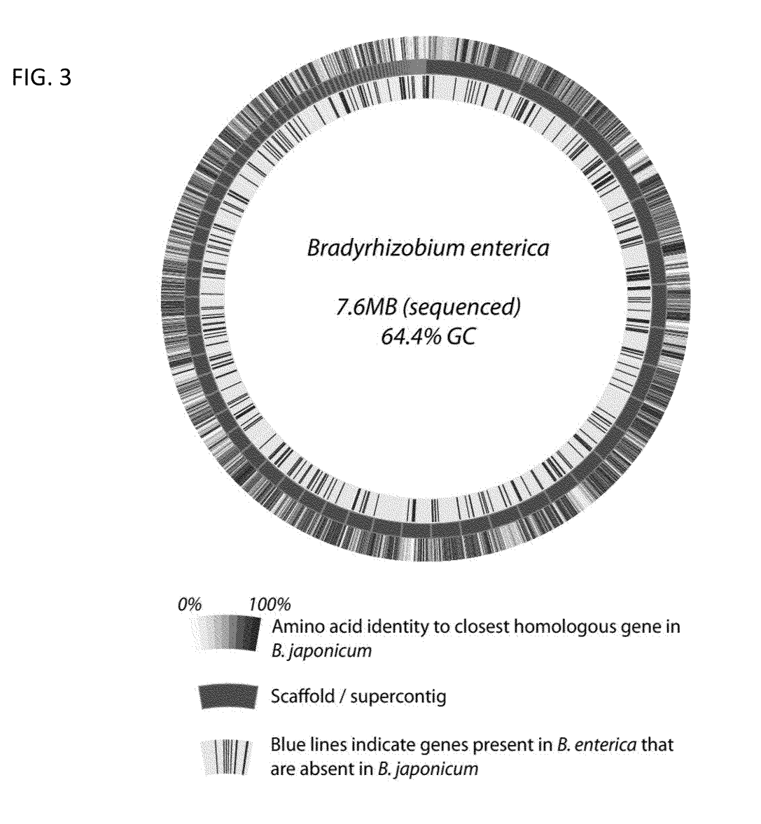

(huttenhower.org/phylophlan), which employs a set of 400 core protein-coding genes in order to generate a rooted phylogenetic tree (FIG. 2). Bootstrap analysis revealed >99% consensus at all branch points except the branch point marked with a circle, where the bootstrap value was 0.181. The organism was provisionally named Bradyrhizobium enterica based on the phylogenetic analysis, which showed a close relationship to Bradyrhizobium japonicum, and the anatomic location of discovery of the organism. The global amino acid sequence identity between homologous B. enterica and B. japonicum proteins (B. japonicum is comprised of a single circular chromosome measuring 9,105,828 bp) is presented in FIG. 3.

Metagenomic Characterization of the Sequenced CCS Samples

In order to determine the proportion of B. enterica to total bacterial reads in the four index samples, PathSeq analysis was carried out once more, with the addition of the draft B. enterica genome to the reference database. The relative abundance of B. enterica reads compared to total quality filtered reads dropped by ˜6.3-fold between the pre-treatment and post-treatment sample (obtained 28 days after antibiotic initiation) in patient 5 and ˜1.7-fold in patient 11 (post-treatment sample obtained 44 days after antibiotic initiation). These relative findings were independently corroborated by PCR. The most abundant bacterial species and selected viruses identified are presented in Table 2a and b. B. enterica was the predominant bacteria in all four samples (Table 2a). In stark contrast to the microbiome of healthy individuals and normal colonic tissue adjacent to colorectal tumors, known intestinal commensals and pathogens, such as Escherichia coli, were present at a much lower abundance than B. enterica (the number of E. coli reads ranging between 0.01 and 0.03% of the total number of reads corresponding to B. enterica). Patient 11 had previously been diagnosed with CMV colitis but had no pathological evidence of viral cytopathic changes at the time of clinical CCS. Of note, the total number of cytomegalovirus (CMV) reads was lower in the second versus the first biopsy in this patient (Table 2b).

Detection of B. enterica in Controls and Additional CCS Patients

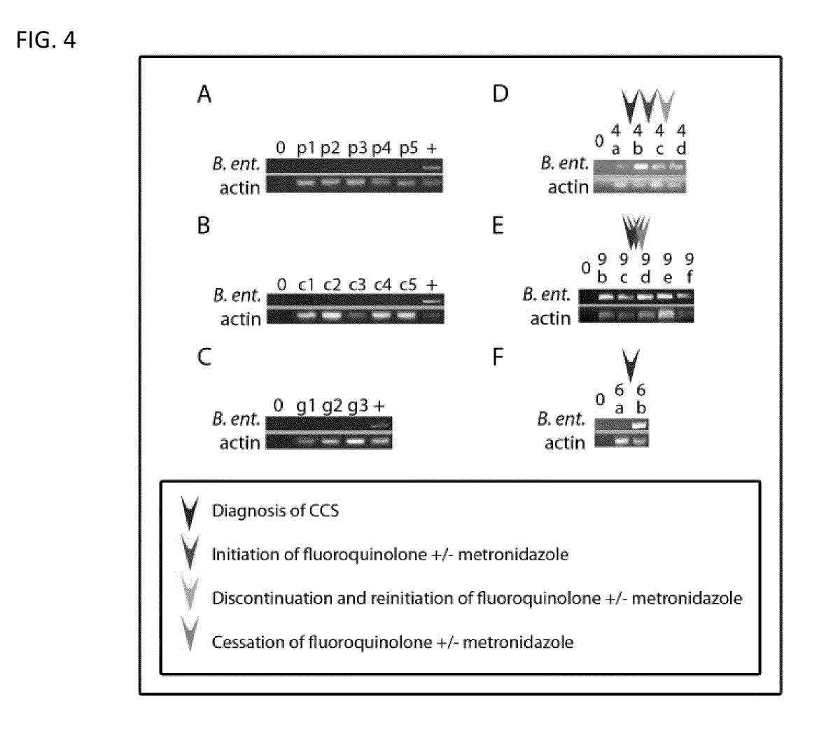

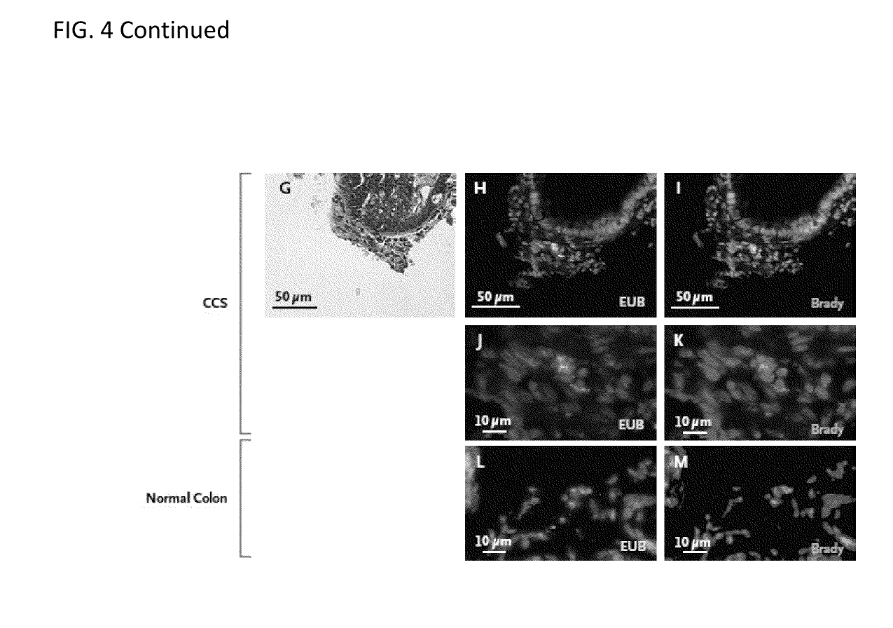

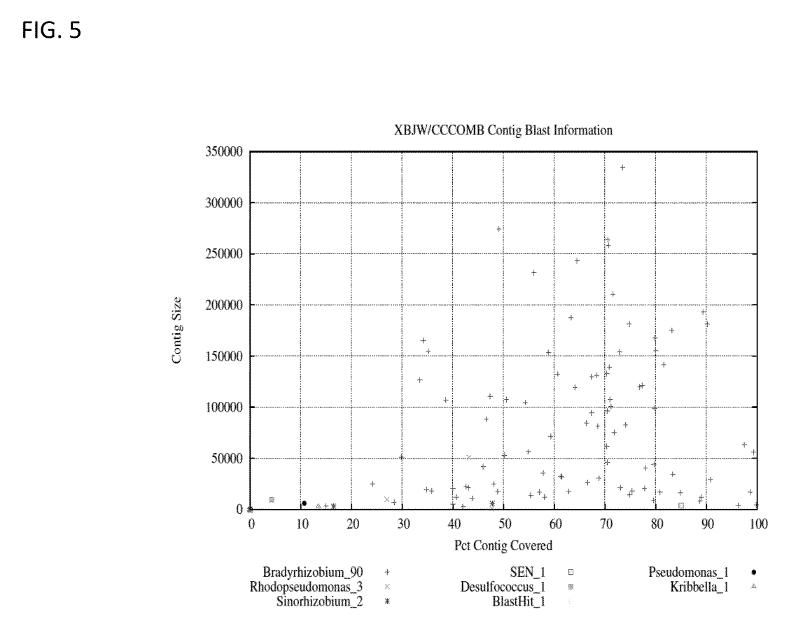

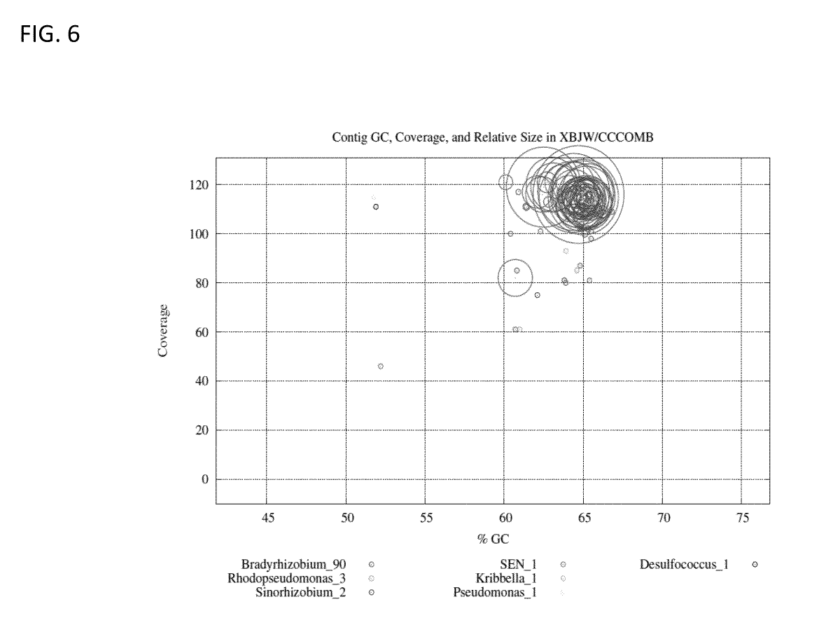

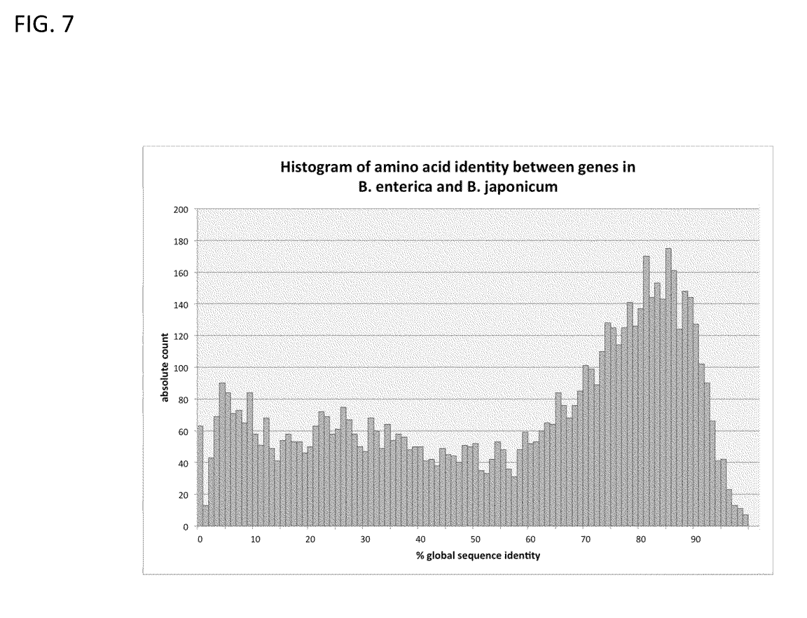

PCR was performed in order to investigate the differential abundance of B. enterica compared to total bacteria and total human cells in CCS patients versus healthy controls, patients with colon cancer, and umbilical cord HSCT patients who carried a pathologically-confirmed diagnosis of GVHD. In addition to these controls, colonic biopsies for three additional patients within 120 days prior to and 200 days after CCS-directed therapy were obtained. Given the very small size of the biopsies and limited sample amount, quantitative studies were not possible. PCR was performed with primers to B. enterica and human actin. The presence of actin in all samples confirms the presence of human tissue within each specimen and the relative intensity of the actin band indicates the relative abundance of actin within the sample. B. enterica was undetectable in all three control tissue types (FIGS. 4A-C). In biopsies from the three additional CCS patients, B. enterica was less abundant in biopsies prior to onset of CCS, was present in all biopsies around the time of diagnosis of CCS, and in some cases, decreased in abundance after CCS treatment with metronidazole +/−fluoroquinolone (FIG. 4D-F).

Conclusion

Conventional microbiological tools are successful in the detection of many clinically significant infectious organisms. Despite this, many potentially infectious syndromes remain idiopathic. Determining a candidate etiological agent in these presumed infectious diseases can be challenging, costly, and is often unsuccessful. Many have predicted that new sensitive and unbiased genomics methods may illuminate candidate etiologic agents in a subset of these diseases, as they have in the past, in selected circumstances.

The present invention demonstrates the discovery of a novel organism, provisionally named Bradyrhizobium enterica, from a cohort of patients with an idiopathic, antibiotic-responsive colitis syndrome using genomic tools. The unusual lack of diversity in the colonic microbiome after HSCT has been described, and the relationships between these altered microbiomes and GVHD and infection are being illuminated. The abundance of B. enterica in these samples suggests that the syndrome is distinct from other known transplantation-associated colitis syndromes. According to the data presented herein, the organism appears to be specific to patients with CCS and is not present in various controls, including patients with intestinal GVHD.

Interestingly, the phylogenetic analysis reveals that B. enterica is taxonomically related to plant endosymbionts such as B. japonicum. Related organisms demonstrate direct or inferred sensitivity of B. enterica to fluoroquinolones, metronidazole, and the therapy that was effective in the treatment of CCS patients from the original cohort. Ferredoxin/pyruvate reductase genes, predicted to have a critical role in the reduction of metronidazole and thus its activity, are present in the draft genome of B. enterica, supporting the conclusion that B. enterica is the therapeutic target of CCS-directed metronidazole based therapy. As B. enterica is not a known pathogen in immunocompetent hosts, it may only be tolerated and cause damage to an immunosuppressed host.

As WGS of human disease specimens becomes more widespread, several novel disease-associated organisms will be discovered using methods similar to those described here.

TABLE 1a
Clinical data regarding antibiotic therapy, temporal and anatomic details of archived gastrointestinal biopsies
in the discovery and validation CCS cohort. Samples in the discovery cohort are indicated by red text in
the “Sample designation” column. Antibiotic therapy is indicated by date; M = metronidazole,
C = ciprofloxacin and L = levofloxacin. Patient nine had an appendectomy several years prior to
transplantation, thus accounting for the pre-transplantation gastrointestinal biopsy specimen.

| | | Diagnosis/ | | | CCS antibiotic therapy (days post SCT) | | | | |
|---|---|---|---|---|---|---|---|---|---|
| | | Transplantation details | | CCS | | | Relapse | | Sample |
| Patient ID | | Diagnosis | | Onset of CCS | Antibiotic | Antibiotic | antibiotic | Relapse | designation |
| Patient # | | (indication | Type of | (days post | start | stop | start | antibiotic | Sample |
| (deidentified) | Gender | for SCT) | SCT | transplantation) | date | date | date | stop | number |
| 4 | F | AML | Myeloablative | 103 | 111 | 121 | 125 | 855 | 4a |
| | | | UC-SCT | | (M, C) | | (M, C) | | 4b |
| | | | | | | | | | 4c |
| | | | | | | | | | 4d |
| | | | | | | | | | 4e |
| 5 | F | CML | Myeloablative | 158 | 181 | 271 | 278 | ongoing | 5a |
| | | | UC-SCT | | (M, C) | | (M, C) | | 5b |
| | | | | | | | | | 5c |
| | | | | | | | | | 5d |
| 6 | M | MDS | Myeloablative | 167 | 177 | 191 | n/a | n/a | 6a |
| | | | UC-SCT | | (M, C) | | | | 6b |
| 9 | M | CLL | RIC UC-SCT | 314 | 375 | 385 | n/a | n/a | 9a |
| | | | | | | | | | 9b |
| | | | | | | | | | 9c |
| | | | | | | | | | 9d |
| | | | | | | | | | 9e |
| | | | | | | | | | 9f |
| | | | | | | | | | 9g |
| | | | | | | | | | 9h |
| 11 | M | HD | RIC UC-SCT | 298 | 298 | 358 | 376 | 436 | 11a |
| | | | | | (M, L) | | (M, L) | | 11b |
| | | | | | | | | | 11c |
| | | | | | | | | | 11d |

| | | GI biopsy date | | | | | | |
|---|---|---|---|---|---|---|---|---|
| | | (days post SCT) | | | | | | |
| Patient ID | | GI biopsy date | | | | | | |
| Patient # | | (with respect | GI biopsy site | | | | | |
| (deidentified) | Gender | to transplant) | Stomach | Duodenum | Ileum | Colon | Sigmoid | Rectum |
| 4 | F | 30 | | | | | x | |
| | | 120 | | | | | x | |
| | | 180 | | | | | x | |
| | | 236 | x | x | | | x | x |
| | | 358 | | x | | x | | |
| 5 | F | 64 | | | | x | | |
| | | 105 | | | | x | | |
| | | 209 | x | x | | | x | |
| | | 526 | x | x | x | x | | |
| 6 | M | 55 | | | | x | | |
| | | 205 | | | x | x | | |
| 9 | M | −5553 | | | | | | |
| | | 257 | | | | | x | |
| | | 312 | x | x | | x | | |
| | | 371 | | | | | x | |
| | | 481 | | | | | x | |
| | | 560 | | | | x | | |
| | | 642 | | | | x | | |
| | | 668 | | | | x | | |
| 11 | M | 205 | | | | x | | |
| | | 266 | x | x | | x | | |
| | | 285 | | | | x | | |
| | | 342 | | x | | | x | |

TABLE 1b
Classification of reads from whole genome shotgun sequencing of formalin-fixed,
paraffin embedded colon biopsy samples from patients with cord colitis syndrome.

| Sample number | 5b | 5c | 11b | 11d |
|---|---|---|---|---|
| Read length | 101 | 101 | 76 | 76 |
| Total number of reads | 134,251,634 | 110,856,860 | 31,045,710 | 41,992,012 |
| Low quality reads (removed) | 52,004,826 | 11,994,589 | 12,688,835 | 17,063,731 |
| Duplicate/repeat reads | 1,625,164 | 2,492,830 | 1,982,166 | 1,351,105 |
| Human reads | 79,951,010 | 96,212,072 | 14,612,284 | 30,119,587 |
| Known bacterial reads | 268,774 | 58,838 | 570,238 | 449,463 |
| Known viral reads | 99* | 125* | 399* | 719* |
| Unmapped reads | 401,761 | 98,406 | 1,191,788 | 955,165 |

Computational analysis of massively parallel DNA sequencing from human tissue samples was performed using PathSeq software. Human reads were computationally subtracted, followed by taxonomic classification with BLASTN to microbial and viral databases. A large proportion of non-human reads were “unmappable” to available reference genomes.

TABLE 1c
Results of contig generation from unmapped read assembly.

| Samples used for assembly | 11b + 11c |
|---|---|
| Number of input reads for assembly* | 4,619,184 |
| Number of contigs (>2.5 kb) | 99 |
| Maximum contig length (bp) | 334,780 |
| Mean contig length (bp) | 77,268 |
| Contig N50 | 141,525 |
| Total Contig length (bp) | 7,649,492 |
| Assembly GC content (% of total bp) | 64.4 |

The ALLPATHS software program was used to assemble unmapped reads from pooled samples (11b and 11d) into longer, contiguous sequences.
*The input reads for assembly were comprised of all non-human reads. All pair-mates of quality filtered reads that were classified as nonhuman were also included in the assembly.

TABLE 2a
Bacterial abundance (in raw read number) of the
27 most abundant bacteria in CCS patients.

| | 5b | 5c | 11b | 11d |
|---|---|---|---|---|
| Organism | number of reads | | | |
| Bradyrhizobium enterica | 631,733 | 119,186 | 1,670,372 | 1,361,453 |
| Delftia acidovorans | 5,028 | 7,532 | 174 | 55 |
| Stenotrophomonas | 2,891 | 3,790 | 200 | 88 |
| maltophilia | | | | |
| Delftia sp. | 2,133 | 2,992 | 472 | 174 |
| Propionibacterium acnes | 1,100 | 362 | 6,045 | 1,101 |
| Bradyrhizobium | 818 | 225 | 1,810 | 1,334 |
| japonicum | | | | |
| Bradyrhizobium sp. | 760 | 165 | 1,512 | 493 |
| Pseudomonas mendocina | 658 | 140 | 1,408 | 1,084 |
| Ralstonia pickettii | 523 | 153 | 1,136 | 771 |
| Rhodopseudomonas | 513 | 83 | 1,549 | 529 |
| palustris | | | | |
| Agrobacterium sp. | 443 | 91 | 207 | 99 |
| Acidovorax ebreus | 233 | 114 | 765 | 331 |
| Agrobacterium | 219 | 115 | 424 | 203 |
| tumefaciens | | | | |
| Streptococcus sanguinis | 214 | 100 | 160 | 274 |
| Rubrivivax gelatinosus | 211 | 196 | 241 | 166 |
| Escherichia coli | 208 | 129 | 256 | 207 |
| Burkholderia gladioli | 204 | 239 | 218 | 115 |
| Pseudomonas fluorescens | 149 | 189 | 72 | 18 |
| Xanthomonas campestris | 109 | 44 | 455 | 269 |
| Fusobacterium | 101 | 127 | 229 | 91 |
| nucleatum | | | | |
| Rhizobium etli | 101 | 36 | 499 | 312 |
| Mesorhizobium | 75 | 84 | 483 | 297 |
| opportunistum | | | | |
| Mesorhizobium loti | 72 | 129 | 15 | 4 |
| Mesorhizobium ciceri | 51 | 18 | 946 | 337 |
| Brucella suis | 28 | 16 | 638 | 181 |
| Pseudomonas putida | 13 | 2 | 174 | 252 |
| Alicycliphilus | 5 | 9 | 603 | 6 |
| denitrificans | | | | |

TABLE 2b
The abundance (number of reads) of a subset
of known human viruses is presented.

| | | | |
|---|---|---|---|
| 5b | 5c | 11b | 11d |

| | |
|---|---|
| Virus | number of reads |

| | | | | |
|---|---|---|---|---|
| TTV | 0 | 10 | 0 | 598 |
| HHV6b | 14 | 46 | 19 | 42 |
| CMV | 0 | 0 | 224 | 7 |
| EBV | 0 | 0 | 0 | 1 |
| KSHV | 0 | 0 | 0 | 1 |
| HHV7 | 2 | 39 | 0 | 0 |

### Example 3

### Additional Methods and Materials

Genome Assembly Methods

Sequencing reads from short fragment sequencing libraries (insert size 150-400 bp) were pooled from temporally separated biopsies from each separate patient (5b+5c and 11b+11d) as well as all four patients (5b+5c+11b+11d). All paired-end sequences were treated as single-end reads and were run through the PathSeq algorithm for computational subtraction of human reads after quality filtering. All non-human reads from these samples and pair-mates of these non-human reads were also included in the assembly, regardless of the quality score of the pair-mate. Two separate computational assembly methods, VELVET1 and ALLPATHS2,3, were employed, as previously described. ALLPATHS was developed as a tool for genome assembly using dual inputs of short fragment sequencing libraries and large fragment (jumping) libraries. In order to use ALLPATHS for assembly, reads were first assembled into a temporary genome. All paired-end reads were aligned using the Burrows-Wheeler alignment algorithm to this temporary genome and insert size was inferred based on alignment of reads pairs. 4,5 Paired-end reads were then split into “shorter” and “longer” fragment pools and were taken forward for formal ALLPATHS assembly. Both assembly methods assembled a total contig length (of contigs >2.5 kb) of greater than 7.5 Mb when applied to the pooled set of reads from all four sequenced samples. The ALLPATHS assembly generated a longer set of contigs for sequences obtained from a single patient and was thus taken forward for further analysis. Given the possibility that the two separate patients harbored slightly different organisms (either at the species or strain level) and the relative similarity of the total contig length generated by the ALLPATHS assembly of sequences from patient 5, this set of 99 contigs was taken forward as the draft genome.

Contig Statistics

### Contigs 99

### Max Contig 334,780

### Mean Contig 77,268

### Contig N50 141,525

### Total Contig Length 7,649,492

### Assembly GC 64.4%

Each contig of greater than 2.5 kb was analyzed for percent GC content and read coverage. Contigs were analyzed by BLASTN6 against the NCBI nt database and were defined by the top hit (that with the lowest E value).

The BLASTN results of each individual contig were evaluated by our genome annotation team (ASB, SSF, SY, DG, AE, BW). The contig corresponding to the SEN virus was determined to be unlikely inserted into the novel organism's genome and was removed from the draft genome. The vast majority of the remaining contigs mapped to members of the family Bradyrhizobaceae and all other contigs mapping to other bacterial families were maintained in the draft genome due to similar coverage and GC content. As there are gaps in the draft genome, there remains the possibility that a small subset of these contigs is not a part of the true B. enterica genome. Future efforts to isolate, culture and complete the genome of this organism will be revealing in this regard, and will also illuminate the question of whether this organism has a circular or linear genome and whether it has a single chromosome or multiple chromosomes.

Contigs were taken forward for further assembly and from the 99 contigs, 90 scaffolds or supercontigs were generated (by end joining of contigs). One of these supercontigs (3,621 bp) corresponded to the SEN virus and was excluded from further analysis.

Scaffold Stats

### Scaffolds 90

### Max Scaffold 533,022

### Mean Scaffold 84,997

### Scaffold N50 155,300

### Total Scaffold Length 7,649,768

SEN virus supercontig length 3,621
Total Scaffold number (minus SEN virus supercontig) 89
Total Scaffold Length (minus SEN virus supercontig) 7,646,147

As the B. enterica genome was assembled from a complex human tissue sample, the genome has been submitted as a “multispecies” sample to the NCBI, as it was not derived from a isolated, purified culture or a true metagenomic sample. The strain has been designated DFCI-1 (Dana-Farber Cancer Institute-1) for the institution and location of care of CCS-affected patients.

Comparative Genomic Analysis and Circos Plot Construction

In order to perform comparative genome analysis of B. enterica, genome annotation was carried out by PRODIGAL (as previously described and cited in the main manuscript). Gene annotations are available on NCBI.

The most closely related species in a phylogenetic analysis reported in the main text was Bradyrhizobium japonicum (strain USDA 110). In order to determine the homology between genes in B. enterica and B. japonicum, each PRODIGAL-predicted gene was compared to the B. japonicum amino acid sequence by peptide BLAST. The full sequence of the top hit was extracted and the full-length genes were then aligned using the Needleman-Wunsch global alignment algorithm. The percentage identity was then calculated for each gene. This value was plotted at the location of the gene on the circular genome plot in the main manuscript. A histogram of global sequence identity by individual gene is provided below.

B. enterica genes for which no homologous B. japonicum gene was identified or for which the global amino acid sequence identity was less than 5% were determined and are plotted in the circular genome plot in the main manuscript. A list of the genes that are specific to B. enterica compared to B. japonicum is below. Note that the PRODIGAL algorithm is a highly specific method that conservatively assigns gene annotations, resulting in a significant number of hypothetical gene “calls”.

TABLE 3
A list of genes present in B. enterica that are absent in B. japonicum
or have homologs with less than 5% identity to B. japonicum.

| | | | Is gene absent in B. |
|---|---|---|---|
| Gene | | amino | japonicum or <5% |
| identification | | acid | identity to a predicted |
| number | Prodigal-predicted gene name | length | B. japonicum homolg? |
| C207_02513 | Bradyrhizobium enterica 2-dehydro-3- | 174 | <5% identity |
| | deoxyphosphogalactonate aldolase | | |
| C207_05358 | Bradyrhizobium enterica 2-haloacid dehalogenase | 50 | <5% identity |
| C207_00881 | Bradyrhizobium enterica 3-oxoacid CoA-transferase | 33 | absent |
| | subunit B | | |
| C207_06559 | Bradyrhizobium enterica 3-oxoacyl-[acyl-carrier-protein] | 70 | <5% identity |
| | synthase III | | |
| C207_01707 | Bradyrhizobium enterica 4-hydroxyacetophenone | 129 | <5% identity |
| | monooxygenase | | |
| C207_02017 | Bradyrhizobium enterica 4-hydroxyphenylacetate-3- | 526 | <5% identity |
| | monooxygenase large chain | | |
| C207_00016 | Bradyrhizobium enterica 6-aminohexanoate-cyclic-dimer | 61 | <5% identity |
| | hydrolase | | |
| C207_06840 | Bradyrhizobium enterica acetoacetyl-CoA synthetase | 48 | <5% identity |
| C207_04517 | Bradyrhizobium enterica alanyl-tRNA synthetase | 48 | <5% identity |
| C207_04847 | Bradyrhizobium enterica alkanesulfonate monooxygenase | 103 | <5% identity |
| C207_04970 | Bradyrhizobium enterica antibiotic transport system ATP- | 71 | <5% identity |
| | binding protein | | |
| C207_02911 | Bradyrhizobium enterica ApaG protein | 49 | <5% identity |
| C207_03323 | Bradyrhizobium enterica aspartate ammonia-lyase | 174 | <5% identity |
| C207_01988 | Bradyrhizobium enterica aspartyl-tRNA (Asn)/glutamyl- | 64 | <5% identity |
| | tRNA (Gln) amidotransferase subunit C | | |
| C207_04254 | Bradyrhizobium enterica ATP-dependent Clp protease | 154 | <5% identity |
| | ATP-binding subunit ClpB | | |
| C207_06703 | Bradyrhizobium enterica ATP-dependent Clp protease | 61 | <5% identity |
| | subunit | | |
| C207_02710 | Bradyrhizobium enterica biopolymer transporter ExbD | 103 | <5% identity |
| C207_02204 | Bradyrhizobium enterica branched-chain amino acid | 112 | <5% identity |
| | transport system ATP-binding protein | | |
| C207_00214 | Bradyrhizobium enterica branched-chain amino acid | 59 | <5% identity |
| | transport system ATP-binding protein | | |
| C207_01742 | Bradyrhizobium enterica branched-chain amino acid | 123 | <5% identity |
| | transport system permease | | |
| C207_02874 | Bradyrhizobium enterica branched-chain amino acid | 338 | <5% identity |
| | transport system substrate-binding protein | | |
| C207_00321 | Bradyrhizobium enterica branched-chain amino acid | 257 | <5% identity |
| | transport system substrate-binding protein | | |
| C207_01678 | Bradyrhizobium enterica carbamate kinase | 320 | <5% identity |
| C207_01177 | Bradyrhizobium enterica CDF family cation efflux system | 155 | <5% identity |
| | protein | | |
| C207_03088 | Bradyrhizobium enterica cell division protein FtsI | 116 | <5% identity |
| | (penicillin-binding protein 3) | | |
| C207_01915 | Bradyrhizobium enterica cobalt transporter subunit CbtB | 64 | <5% identity |
| | (proposed) | | |
| C207_00003 | Bradyrhizobium enterica cobalt-precorrin 5A hydrolase | 138 | <5% identity |
| C207_05190 | Bradyrhizobium enterica cytochrome d ubiquinol oxidase | 633 | <5% identity |
| | subunit II | | |
| C207_06585 | Bradyrhizobium enterica D-threo-aldose 1-dehydrogenase | 84 | <5% identity |
| C207_01857 | Bradyrhizobium enterica DNA repair protein RecN | 66 | <5% identity |
| | (Recombination protein N) | | |
| C207_02942 | Bradyrhizobium enterica DNA-3-methyladenine | 63 | <5% identity |
| | glycosylase II | | |
| C207_00234 | Bradyrhizobium enterica DOPA 4,5-dioxygenase | 137 | <5% identity |
| C207_01169 | Bradyrhizobium enterica dTDP-4-dehydrorhamnose 3,5- | 111 | <5% identity |
| | epimerase | | |
| C207_00459 | Bradyrhizobium enterica FdhD protein | 140 | <5% identity |
| C207_06358 | Bradyrhizobium enterica Fe—S cluster assembly protein | 67 | <5% identity |
| | SufD | | |
| C207_03698 | Bradyrhizobium enterica filamentous hemagglutinin | 4428 | <5% identity |
| | family domain-containing protein | | |
| C207_01723 | Bradyrhizobium enterica filamentous hemagglutinin | 4282 | <5% identity |
| | family domain-containing protein | | |
| C207_01969 | Bradyrhizobium enterica filamentous hemagglutinin | 4010 | <5% identity |
| | family domain-containing protein | | |
| C207_04905 | Bradyrhizobium enterica filamentous hemagglutinin | 3769 | <5% identity |
| | family domain-containing protein | | |
| C207_02878 | Bradyrhizobium enterica flagellum-specific ATP synthase | 226 | <5% identity |
| C207_04305 | Bradyrhizobium enterica formyl-CoA transferase | 127 | <5% identity |
| C207_05832 | Bradyrhizobium enterica galactarate dehydratase | 82 | <5% identity |
| C207_02559 | Bradyrhizobium enterica general secretion pathway | 104 | <5% identity |
| | protein D | | |
| C207_07133 | Bradyrhizobium enterica glutathione transport system | 148 | <5% identity |
| | permease | | |
| C207_00007 | Bradyrhizobium enterica glycerol-3-phosphate | 895 | <5% identity |
| | dehydrogenase | | |
| C207_06841 | Bradyrhizobium enterica haloacetate dehalogenase | 46 | <5% identity |
| C207_07098 | Bradyrhizobium enterica hypothetical protein | 2910 | <5% identity |
| C207_06833 | Bradyrhizobium enterica hypothetical protein | 1855 | <5% identity |
| C207_06429 | Bradyrhizobium enterica hypothetical protein | 816 | <5% identity |
| C207_01070 | Bradyrhizobium enterica hypothetical protein | 599 | <5% identity |
| C207_02136 | Bradyrhizobium enterica hypothetical protein | 587 | <5% identity |
| C207_03202 | Bradyrhizobium enterica hypothetical protein | 545 | absent |
| C207_01463 | Bradyrhizobium enterica hypothetical protein | 543 | <5% identity |
| C207_03999 | Bradyrhizobium enterica hypothetical protein | 463 | <5% identity |
| C207_02931 | Bradyrhizobium enterica hypothetical protein | 437 | absent |
| C207_01798 | Bradyrhizobium enterica hypothetical protein | 431 | <5% identity |
| C207_05230 | Bradyrhizobium enterica hypothetical protein | 430 | <5% identity |
| C207_06785 | Bradyrhizobium enterica hypothetical protein | 415 | <5% identity |
| C207_01242 | Bradyrhizobium enterica hypothetical protein | 382 | <5% identity |
| C207_02843 | Bradyrhizobium enterica hypothetical protein | 366 | absent |
| C207_02120 | Bradyrhizobium enterica hypothetical protein | 334 | <5% identity |
| C207_04081 | Bradyrhizobium enterica hypothetical protein | 334 | <5% identity |
| C207_03341 | Bradyrhizobium enterica hypothetical protein | 327 | <5% identity |
| C207_07094 | Bradyrhizobium enterica hypothetical protein | 294 | <5% identity |
| C207_05219 | Bradyrhizobium enterica hypothetical protein | 288 | absent |
| C207_03599 | Bradyrhizobium enterica hypothetical protein | 283 | <5% identity |
| C207_01150 | Bradyrhizobium enterica hypothetical protein | 259 | <5% identity |
| C207_05854 | Bradyrhizobium enterica hypothetical protein | 255 | <5% identity |
| C207_00970 | Bradyrhizobium enterica hypothetical protein | 233 | <5% identity |
| C207_01966 | Bradyrhizobium enterica hypothetical protein | 225 | <5% identity |
| C207_06967 | Bradyrhizobium enterica hypothetical protein | 225 | <5% identity |
| C207_05333 | Bradyrhizobium enterica hypothetical protein | 206 | <5% identity |
| C207_06540 | Bradyrhizobium enterica hypothetical protein | 197 | <5% identity |
| C207_05378 | Bradyrhizobium enterica hypothetical protein | 196 | <5% identity |
| C207_01191 | Bradyrhizobium enterica hypothetical protein | 196 | absent |
| C207_06786 | Bradyrhizobium enterica hypothetical protein | 186 | absent |
| C207_00400 | Bradyrhizobium enterica hypothetical protein | 183 | <5% identity |
| C207_03995 | Bradyrhizobium enterica hypothetical protein | 176 | <5% identity |
| C207_02696 | Bradyrhizobium enterica hypothetical protein | 174 | <5% identity |
| C207_01068 | Bradyrhizobium enterica hypothetical protein | 160 | <5% identity |
| C207_01535 | Bradyrhizobium enterica hypothetical protein | 157 | <5% identity |
| C207_03228 | Bradyrhizobium enterica hypothetical protein | 152 | <5% identity |
| C207_04620 | Bradyrhizobium enterica hypothetical protein | 151 | <5% identity |
| C207_07089 | Bradyrhizobium enterica hypothetical protein | 146 | <5% identity |
| C207_01330 | Bradyrhizobium enterica hypothetical protein | 145 | <5% identity |
| C207_00316 | Bradyrhizobium enterica hypothetical protein | 144 | <5% identity |
| C207_06454 | Bradyrhizobium enterica hypothetical protein | 143 | <5% identity |
| C207_06934 | Bradyrhizobium enterica hypothetical protein | 140 | <5% identity |
| C207_06065 | Bradyrhizobium enterica hypothetical protein | 137 | <5% identity |
| C207_06412 | Bradyrhizobium enterica hypothetical protein | 130 | absent |
| C207_04600 | Bradyrhizobium enterica hypothetical protein | 128 | <5% identity |
| C207_02656 | Bradyrhizobium enterica hypothetical protein | 126 | <5% identity |
| C207_06022 | Bradyrhizobium enterica hypothetical protein | 125 | <5% identity |
| C207_00934 | Bradyrhizobium enterica hypothetical protein | 121 | <5% identity |
| C207_05116 | Bradyrhizobium enterica hypothetical protein | 121 | <5% identity |
| C207_05441 | Bradyrhizobium enterica hypothetical protein | 120 | <5% identity |
| C207_04449 | Bradyrhizobium enterica hypothetical protein | 119 | <5% identity |
| C207_06118 | Bradyrhizobium enterica hypothetical protein | 118 | <5% identity |
| C207_05934 | Bradyrhizobium enterica hypothetical protein | 115 | <5% identity |
| C207_01403 | Bradyrhizobium enterica hypothetical protein | 113 | <5% identity |
| C207_03963 | Bradyrhizobium enterica hypothetical protein | 111 | <5% identity |
| C207_05797 | Bradyrhizobium enterica hypothetical protein | 108 | <5% identity |
| C207_00550 | Bradyrhizobium enterica hypothetical protein | 106 | <5% identity |
| C207_04611 | Bradyrhizobium enterica hypothetical protein | 106 | <5% identity |
| C207_01406 | Bradyrhizobium enterica hypothetical protein | 105 | <5% identity |
| C207_01734 | Bradyrhizobium enterica hypothetical protein | 105 | <5% identity |
| C207_02902 | Bradyrhizobium enterica hypothetical protein | 105 | <5% identity |
| C207_04614 | Bradyrhizobium enterica hypothetical protein | 105 | <5% identity |
| C207_05167 | Bradyrhizobium enterica hypothetical protein | 104 | <5% identity |
| C207_01791 | Bradyrhizobium enterica hypothetical protein | 103 | <5% identity |
| C207_05570 | Bradyrhizobium enterica hypothetical protein | 103 | <5% identity |
| C207_01794 | Bradyrhizobium enterica hypothetical protein | 102 | <5% identity |
| C207_03993 | Bradyrhizobium enterica hypothetical protein | 100 | <5% identity |
| C207_04236 | Bradyrhizobium enterica hypothetical protein | 100 | <5% identity |
| C207_06932 | Bradyrhizobium enterica hypothetical protein | 100 | <5% identity |
| C207_06922 | Bradyrhizobium enterica hypothetical protein | 99 | <5% identity |
| C207_06562 | Bradyrhizobium enterica hypothetical protein | 98 | <5% identity |
| C207_05824 | Bradyrhizobium enterica hypothetical protein | 97 | <5% identity |
| C207_06950 | Bradyrhizobium enterica hypothetical protein | 96 | <5% identity |
| C207_04264 | Bradyrhizobium enterica hypothetical protein | 94 | <5% identity |
| C207_06139 | Bradyrhizobium enterica hypothetical protein | 94 | <5% identity |
| C207_01751 | Bradyrhizobium enterica hypothetical protein | 93 | <5% identity |
| C207_03614 | Bradyrhizobium enterica hypothetical protein | 90 | <5% identity |
| C207_04833 | Bradyrhizobium enterica hypothetical protein | 90 | <5% identity |
| C207_06299 | Bradyrhizobium enterica hypothetical protein | 89 | <5% identity |
| C207_01183 | Bradyrhizobium enterica hypothetical protein | 88 | <5% identity |
| C207_01430 | Bradyrhizobium enterica hypothetical protein | 88 | <5% identity |
| C207_02833 | Bradyrhizobium enterica hypothetical protein | 88 | <5% identity |
| C207_04597 | Bradyrhizobium enterica hypothetical protein | 88 | <5% identity |
| C207_03399 | Bradyrhizobium enterica hypothetical protein | 88 | absent |
| C207_04481 | Bradyrhizobium enterica hypothetical protein | 87 | <5% identity |
| C207_06845 | Bradyrhizobium enterica hypothetical protein | 87 | <5% identity |
| C207_01212 | Bradyrhizobium enterica hypothetical protein | 86 | <5% identity |
| C207_01529 | Bradyrhizobium enterica hypothetical protein | 86 | <5% identity |
| C207_07126 | Bradyrhizobium enterica hypothetical protein | 86 | <5% identity |
| C207_01077 | Bradyrhizobium enterica hypothetical protein | 85 | <5% identity |
| C207_01552 | Bradyrhizobium enterica hypothetical protein | 85 | <5% identity |
| C207_02712 | Bradyrhizobium enterica hypothetical protein | 85 | <5% identity |
| C207_03892 | Bradyrhizobium enterica hypothetical protein | 85 | <5% identity |
| C207_04468 | Bradyrhizobium enterica hypothetical protein | 85 | <5% identity |
| C207_03949 | Bradyrhizobium enterica hypothetical protein | 84 | <5% identity |
| C207_04454 | Bradyrhizobium enterica hypothetical protein | 83 | <5% identity |
| C207_05444 | Bradyrhizobium enterica hypothetical protein | 82 | <5% identity |
| C207_00280 | Bradyrhizobium enterica hypothetical protein | 81 | <5% identity |
| C207_05913 | Bradyrhizobium enterica hypothetical protein | 80 | <5% identity |
| C207_04376 | Bradyrhizobium enterica hypothetical protein | 79 | <5% identity |
| C207_01418 | Bradyrhizobium enterica hypothetical protein | 78 | <5% identity |
| C207_02008 | Bradyrhizobium enterica hypothetical protein | 78 | <5% identity |
| C207_06615 | Bradyrhizobium enterica hypothetical protein | 78 | <5% identity |
| C207_06707 | Bradyrhizobium enterica hypothetical protein | 78 | <5% identity |
| C207_00504 | Bradyrhizobium enterica hypothetical protein | 75 | <5% identity |
| C207_04314 | Bradyrhizobium enterica hypothetical protein | 75 | <5% identity |
| C207_05933 | Bradyrhizobium enterica hypothetical protein | 74 | <5% identity |
| C207_06935 | Bradyrhizobium enterica hypothetical protein | 74 | <5% identity |
| C207_07049 | Bradyrhizobium enterica hypothetical protein | 73 | absent |
| C207_00413 | Bradyrhizobium enterica hypothetical protein | 72 | <5% identity |
| C207_05375 | Bradyrhizobium enterica hypothetical protein | 72 | <5% identity |
| C207_05382 | Bradyrhizobium enterica hypothetical protein | 72 | <5% identity |
| C207_06111 | Bradyrhizobium enterica hypothetical protein | 72 | <5% identity |
| C207_00150 | Bradyrhizobium enterica hypothetical protein | 71 | <5% identity |
| C207_00595 | Bradyrhizobium enterica hypothetical protein | 70 | <5% identity |
| C207_01078 | Bradyrhizobium enterica hypothetical protein | 69 | <5% identity |
| C207_04557 | Bradyrhizobium enterica hypothetical protein | 69 | <5% identity |
| C207_06134 | Bradyrhizobium enterica hypothetical protein | 69 | <5% identity |
| C207_02575 | Bradyrhizobium enterica hypothetical protein | 67 | <5% identity |
| C207_03144 | Bradyrhizobium enterica hypothetical protein | 67 | <5% identity |
| C207_05053 | Bradyrhizobium enterica hypothetical protein | 67 | <5% identity |
| C207_02003 | Bradyrhizobium enterica hypothetical protein | 67 | absent |
| C207_01786 | Bradyrhizobium enterica hypothetical protein | 66 | <5% identity |
| C207_03465 | Bradyrhizobium enterica hypothetical protein | 65 | <5% identity |
| C207_04529 | Bradyrhizobium enterica hypothetical protein | 65 | absent |
| C207_04394 | Bradyrhizobium enterica hypothetical protein | 64 | <5% identity |
| C207_05648 | Bradyrhizobium enterica hypothetical protein | 64 | <5% identity |
| C207_05858 | Bradyrhizobium enterica hypothetical protein | 64 | <5% identity |
| C207_01792 | Bradyrhizobium enterica hypothetical protein | 63 | <5% identity |
| C207_04266 | Bradyrhizobium enterica hypothetical protein | 63 | <5% identity |
| C207_04616 | Bradyrhizobium enterica hypothetical protein | 63 | <5% identity |
| C207_06462 | Bradyrhizobium enterica hypothetical protein | 63 | <5% identity |
| C207_03940 | Bradyrhizobium enterica hypothetical protein | 63 | absent |
| C207_00554 | Bradyrhizobium enterica hypothetical protein | 62 | <5% identity |
| C207_01735 | Bradyrhizobium enterica hypothetical protein | 61 | <5% identity |
| C207_07090 | Bradyrhizobium enterica hypothetical protein | 61 | <5% identity |
| C207_03964 | Bradyrhizobium enterica hypothetical protein | 60 | <5% identity |
| C207_00944 | Bradyrhizobium enterica hypothetical protein | 59 | <5% identity |
| C207_02529 | Bradyrhizobium enterica hypothetical protein | 59 | <5% identity |
| C207_05937 | Bradyrhizobium enterica hypothetical protein | 59 | <5% identity |
| C207_01419 | Bradyrhizobium enterica hypothetical protein | 58 | <5% identity |
| C207_05381 | Bradyrhizobium enterica hypothetical protein | 58 | <5% identity |
| C207_07127 | Bradyrhizobium enterica hypothetical protein | 58 | <5% identity |
| C207_03618 | Bradyrhizobium enterica hypothetical protein | 56 | <5% identity |
| C207_04383 | Bradyrhizobium enterica hypothetical protein | 56 | <5% identity |
| C207_04477 | Bradyrhizobium enterica hypothetical protein | 56 | <5% identity |
| C207_06492 | Bradyrhizobium enterica hypothetical protein | 56 | <5% identity |
| C207_01534 | Bradyrhizobium enterica hypothetical protein | 55 | <5% identity |
| C207_02125 | Bradyrhizobium enterica hypothetical protein | 55 | <5% identity |
| C207_02301 | Bradyrhizobium enterica hypothetical protein | 55 | <5% identity |
| C207_03151 | Bradyrhizobium enterica hypothetical protein | 55 | <5% identity |
| C207_05406 | Bradyrhizobium enterica hypothetical protein | 55 | <5% identity |
| C207_02339 | Bradyrhizobium enterica hypothetical protein | 55 | absent |
| C207_02516 | Bradyrhizobium enterica hypothetical protein | 54 | <5% identity |
| C207_01148 | Bradyrhizobium enterica hypothetical protein | 54 | absent |
| C207_01785 | Bradyrhizobium enterica hypothetical protein | 53 | <5% identity |
| C207_05735 | Bradyrhizobium enterica hypothetical protein | 53 | <5% identity |
| C207_01140 | Bradyrhizobium enterica hypothetical protein | 52 | <5% identity |
| C207_04958 | Bradyrhizobium enterica hypothetical protein | 52 | <5% identity |
| C207_00534 | Bradyrhizobium enterica hypothetical protein | 52 | absent |
| C207_00542 | Bradyrhizobium enterica hypothetical protein | 51 | <5% identity |
| C207_02937 | Bradyrhizobium enterica hypothetical protein | 51 | <5% identity |
| C207_01699 | Bradyrhizobium enterica hypothetical protein | 50 | <5% identity |
| C207_05214 | Bradyrhizobium enterica hypothetical protein | 50 | <5% identity |
| C207_01531 | Bradyrhizobium enterica hypothetical protein | 50 | absent |
| C207_06225 | Bradyrhizobium enterica hypothetical protein | 50 | absent |
| C207_02152 | Bradyrhizobium enterica hypothetical protein | 49 | <5% identity |
| C207_05285 | Bradyrhizobium enterica hypothetical protein | 48 | <5% identity |
| C207_00588 | Bradyrhizobium enterica hypothetical protein | 48 | absent |
| C207_00722 | Bradyrhizobium enterica hypothetical protein | 48 | absent |
| C207_05494 | Bradyrhizobium enterica hypothetical protein | 48 | absent |
| C207_05703 | Bradyrhizobium enterica hypothetical protein | 47 | <5% identity |
| C207_01554 | Bradyrhizobium enterica hypothetical protein | 47 | absent |
| C207_01097 | Bradyrhizobium enterica hypothetical protein | 46 | <5% identity |
| C207_00969 | Bradyrhizobium enterica hypothetical protein | 45 | <5% identity |
| C207_01800 | Bradyrhizobium enterica hypothetical protein | 45 | <5% identity |
| C207_00634 | Bradyrhizobium enterica hypothetical protein | 45 | absent |
| C207_04590 | Bradyrhizobium enterica hypothetical protein | 45 | absent |
| C207_05503 | Bradyrhizobium enterica hypothetical protein | 45 | absent |
| C207_04915 | Bradyrhizobium enterica hypothetical protein | 43 | <5% identity |
| C207_05938 | Bradyrhizobium enterica hypothetical protein | 43 | <5% identity |
| C207_06288 | Bradyrhizobium enterica hypothetical protein | 43 | <5% identity |
| C207_06994 | Bradyrhizobium enterica hypothetical protein | 43 | <5% identity |
| C207_01562 | Bradyrhizobium enterica hypothetical protein | 43 | absent |
| C207_02714 | Bradyrhizobium enterica hypothetical protein | 42 | <5% identity |
| C207_05112 | Bradyrhizobium enterica hypothetical protein | 42 | <5% identity |
| C207_01514 | Bradyrhizobium enterica hypothetical protein | 40 | <5% identity |
| C207_00810 | Bradyrhizobium enterica hypothetical protein | 40 | absent |
| C207_01141 | Bradyrhizobium enterica hypothetical protein | 40 | absent |
| C207_06362 | Bradyrhizobium enterica hypothetical protein | 38 | <5% identity |
| C207_04129 | Bradyrhizobium enterica hypothetical protein | 38 | absent |
| C207_01374 | Bradyrhizobium enterica hypothetical protein | 37 | absent |
| C207_04916 | Bradyrhizobium enterica hypothetical protein | 37 | absent |
| C207_05693 | Bradyrhizobium enterica hypothetical protein | 37 | absent |
| C207_01743 | Bradyrhizobium enterica hypothetical protein | 36 | <5% identity |
| C207_04602 | Bradyrhizobium enterica hypothetical protein | 36 | <5% identity |
| C207_01872 | Bradyrhizobium enterica hypothetical protein | 36 | absent |
| C207_02486 | Bradyrhizobium enterica hypothetical protein | 36 | absent |
| C207_04189 | Bradyrhizobium enterica hypothetical protein | 36 | absent |
| C207_04202 | Bradyrhizobium enterica hypothetical protein | 36 | absent |
| C207_03601 | Bradyrhizobium enterica hypothetical protein | 35 | absent |
| C207_00591 | Bradyrhizobium enterica hypothetical protein | 34 | <5% identity |
| C207_04350 | Bradyrhizobium enterica hypothetical protein | 34 | absent |
| C207_06503 | Bradyrhizobium enterica hypothetical protein | 34 | absent |
| C207_06891 | Bradyrhizobium enterica hypothetical protein | 34 | absent |
| C207_00911 | Bradyrhizobium enterica hypothetical protein | 33 | <5% identity |
| C207_01059 | Bradyrhizobium enterica hypothetical protein | 33 | absent |
| C207_04209 | Bradyrhizobium enterica hypothetical protein | 33 | absent |
| C207_05684 | Bradyrhizobium enterica hypothetical protein | 33 | absent |
| C207_06783 | Bradyrhizobium enterica hypothetical protein | 32 | <5% identity |
| C207_02248 | Bradyrhizobium enterica hypothetical protein | 32 | absent |
| C207_03468 | Bradyrhizobium enterica hypothetical protein | 32 | absent |
| C207_06482 | Bradyrhizobium enterica hypothetical protein | 32 | absent |
| C207_07159 | Bradyrhizobium enterica hypothetical protein | 32 | absent |
| C207_03294 | Bradyrhizobium enterica hypothetical protein | 31 | absent |
| C207_00015 | Bradyrhizobium enterica hypothetical protein | 30 | absent |
| C207_00039 | Bradyrhizobium enterica hypothetical protein | 30 | absent |
| C207_01058 | Bradyrhizobium enterica hypothetical protein | 30 | absent |
| C207_01698 | Bradyrhizobium enterica hypothetical protein | 30 | absent |
| C207_01804 | Bradyrhizobium enterica hypothetical protein | 29 | absent |
| C207_03018 | Bradyrhizobium enterica hypothetical protein | 23 | absent |
| C207_06871 | Bradyrhizobium enterica hypothetical protein | 20 | absent |
| C207_03997 | Bradyrhizobium enterica indolepyruvate ferredoxin | 166 | <5% identity |
| | oxidoreductase | | |
| C207_02900 | Bradyrhizobium enterica light-harvesting protein B-880 | 64 | <5% identity |
| | alpha chain | | |
| C207_02901 | Bradyrhizobium enterica light-harvesting protein B-880 | 73 | <5% identity |
| | beta chain | | |
| C207_06138 | Bradyrhizobium enterica lipid A biosynthesis lauroyl | 256 | <5% identity |
| | acyltransferase | | |
| C207_04704 | Bradyrhizobium enterica long-chain acyl-CoA synthetase | 71 | <5% identity |
| C207_01846 | Bradyrhizobium enterica magnesium transporter | 51 | <5% identity |
| C207_00945 | Bradyrhizobium enterica malate dehydrogenase | 81 | <5% identity |
| | (oxaloacetate-decarboxylating) | | |
| C207_01039 | Bradyrhizobium enterica membrane protein | 179 | <5% identity |
| C207_04947 | Bradyrhizobium enterica membrane-bound serine protease | 174 | <5% identity |
| | (ClpP class) | | |
| C207_02895 | Bradyrhizobium enterica MFS transporter, BCD family, | 452 | <5% identity |
| | chlorophyll transporter | | |
| C207_03996 | Bradyrhizobium enterica MFS transporter, BCD family, | 168 | <5% identity |
| | chlorophyll transporter | | |
| C207_03721 | Bradyrhizobium enterica MFS transporter, BCD family, | 158 | <5% identity |
| | chlorophyll transporter | | |
| C207_02016 | Bradyrhizobium enterica muconolactone delta-isomerase | 97 | <5% identity |
| C207_01524 | Bradyrhizobium enterica multidrug efflux transporter | 69 | absent |
| | MdtA | | |
| C207_06828 | Bradyrhizobium enterica multiple sugar transport system | 174 | <5% identity |
| | substrate-binding protein | | |
| C207_03140 | Bradyrhizobium enterica NAD(P) transhydrogenase | 186 | <5% identity |
| | subunit beta | | |
| C207_07161 | Bradyrhizobium enterica nitrite reductase (NAD(P)H) | 64 | absent |
| | large subunit | | |
| C207_00317 | Bradyrhizobium enterica NitT/TauT family transport | 86 | <5% identity |
| | system ATP-binding protein | | |
| C207_03397 | Bradyrhizobium enterica oxidoreductase | 155 | <5% identity |
| C207_04149 | Bradyrhizobium enterica penicillin-binding protein 1A | 343 | <5% identity |
| C207_03586 | Bradyrhizobium enterica peptide/nickel transport system | 61 | absent |
| | permease | | |
| C207_04636 | Bradyrhizobium enterica periplasmic protein TonB | 55 | <5% identity |
| C207_04848 | Bradyrhizobium enterica permease | 104 | <5% identity |
| C207_04512 | Bradyrhizobium enterica phosphinothricin | 222 | <5% identity |
| | acetyltransferase | | |
| C207_00961 | Bradyrhizobium enterica phosphoglycolate phosphatase | 72 | <5% identity |
| C207_05411 | Bradyrhizobium enterica phytoene synthase | 65 | <5% identity |
| C207_01174 | Bradyrhizobium enterica protease | 492 | <5% identity |
| C207_02899 | Bradyrhizobium enterica reaction center protein L chain | 279 | absent |
| C207_02763 | Bradyrhizobium enterica RelE/StbE family addiction | 99 | <5% identity |
| | module toxin | | |
| C207_05676 | Bradyrhizobium enterica ribose 5-phosphate isomerase A | 52 | absent |
| C207_03424 | Bradyrhizobium enterica simple sugar transport system | 62 | <5% identity |
| | ATP-binding protein | | |
| C207_05162 | Bradyrhizobium enterica small GTP-binding protein | 171 | <5% identity |
| C207_03318 | Bradyrhizobium enterica starch synthase | 64 | <5% identity |
| C207_05284 | Bradyrhizobium enterica starvation-inducible DNA- | 438 | absent |
| | binding protein | | |
| C207_00516 | Bradyrhizobium enterica sulfonate transport system | 670 | <5% identity |
| | substrate-binding protein | | |
| C207_06059 | Bradyrhizobium enterica tat (twin-arginine translocation) | 101 | <5% identity |
| | pathway signal sequence | | |
| C207_05879 | Bradyrhizobium enterica threonine synthase | 238 | <5% identity |
| C207_02700 | Bradyrhizobium enterica TonB family domain-containing | 237 | <5% identity |
| | protein | | |
| C207_05118 | Bradyrhizobium enterica transcriptional regulator | 73 | <5% identity |
| C207_03226 | Bradyrhizobium enterica transmembrane sensor | 211 | <5% identity |
| C207_05463 | Bradyrhizobium enterica two-component system, | 42 | <5% identity |
| | chemotaxis family, sensor kinase CheA | | |
| C207_06398 | Bradyrhizobium enterica two-component system, | 31 | <5% identity |
| | chemotaxis family, sensor kinase CheA | | |
| C207_06941 | Bradyrhizobium enterica two-component system, | 31 | <5% identity |
| | chemotaxis family, sensor kinase CheA | | |
| C207_07005 | Bradyrhizobium enterica two-component system, | 31 | <5% identity |
| | chemotaxis family, sensor kinase CheA | | |
| C207_07110 | Bradyrhizobium enterica two-component system, OmpR | 137 | <5% identity |
| | family, phosphate regulon response regulator OmpR | | |
| C207_04538 | Bradyrhizobium enterica type IV secretion system protein | 99 | <5% identity |
| | VirB2 | | |
| C207_00251 | Bradyrhizobium enterica UDPglucose 6-dehydrogenase | 99 | <5% identity |
| C207_03021 | Bradyrhizobium enterica urease accessory protein ureE | 204 | <5% identity |
| C207_06301 | Bradyrhizobium enterica uroporphyrinogen-III synthase | 92 | <5% identity |
| C207_04870 | Bradyrhizobium enterica YD repeat (two copies) | 63 | <5% identity |

Contamination Analysis

Several limitations are introduced by the execution of a single center study that may increase the likelihood of contamination including (1) common paraffin baths used for the generation of FFPE samples, (2) a common nosocomial microbiome, (3) FFPE block handling by a single laboratory, (4) preparation of libraries using very limited DNA in a single laboratory location.

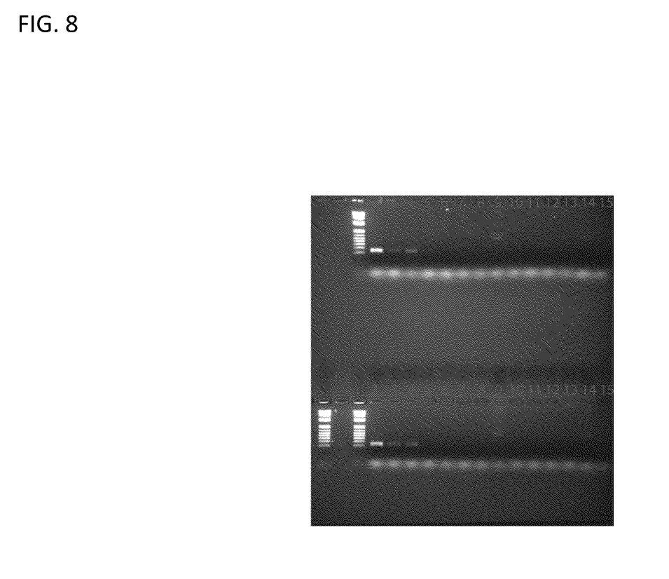

The experimental method employed in this single-center study was designed to minimize the likelihood that the results obtained were due to a contaminant as follows: (1) FFPE colon biopsy samples from normal controls and post-stem cell transplantation GVHD controls processed at the same institution were included and did not demonstrate appreciable B. enterica by PCR. (2) Additional frozen colon cancer controls were also included in this analysis and did not demonstrate appreciable B. enterica by PCR. (3) DNA extraction for the samples that were sequenced was started on the same day but was completed on successive days. (4) Two different type of barcodes generated at different facilities were used to generate sequencing libraries. (5) Samples 5b+5c and 11b+11d were sequenced at two different sequencing facilities. (6) Buffers and ultrapure water used in the extraction of DNA and generation of the libraries were subjected to targeted PCR to investigate for B. enterica in the stock solutions used (FIG. 8). (7) DNA extraction and sequencing library construction was carried out in a dedicated “clean facility” away from lab areas where organisms are cultured. (8) As samples were very limited, the reserved “top scrolls” from two of the samples (9d and 9e) were subjected to DNA extraction several months after the original extraction and B. enterica was present in both scrolls that were studied (FIG. 8). (10) All FFPE samples prepared for sequencing in our laboratory within four months of the CCS samples were analyzed by PathSeq for the presence of B. enterica. Single nucleotide polymorphism analysis was limited by the reported intrinsic low polymorphism rate of organisms such as Bradyrhizobium japonicum USDA 110 and relatively low coverage of B. enterica for samples 5b+5c. Despite this, it appeared that there were at least five to 11 SNPs at an allelic fraction of at least 40% between B. enterica reads from patient 5 vs. patient 11. Additional intrinsic difficulties in evaluation for SNPs include the lack of a completed genome and the high GC content of the organism, which can lead to more frequent sequencing errors.

PCR Conditions

PCR was performed using 10 μM forward and reverse primers, 0.2 ng of input DNA and the AccuPrime Taq DNA polymerase system (Invitrogen, Grand Island, N.Y., USA) per manufacturer's directions in a total volume of 10 μl with the following cycle protocol: 95° C. for 2 minutes, followed by 35 cycles of: 95° C. for 30 seconds, 62.1° C. for 30 seconds, 68° C. for 40 seconds, and finally an extension at 68° C. for 5 minutes. PCR was carried out on an Eppendorf AG Mastercycler Pro (Hauppauge, N.Y., USA).

Viral Reads in Sequenced CCS Samples

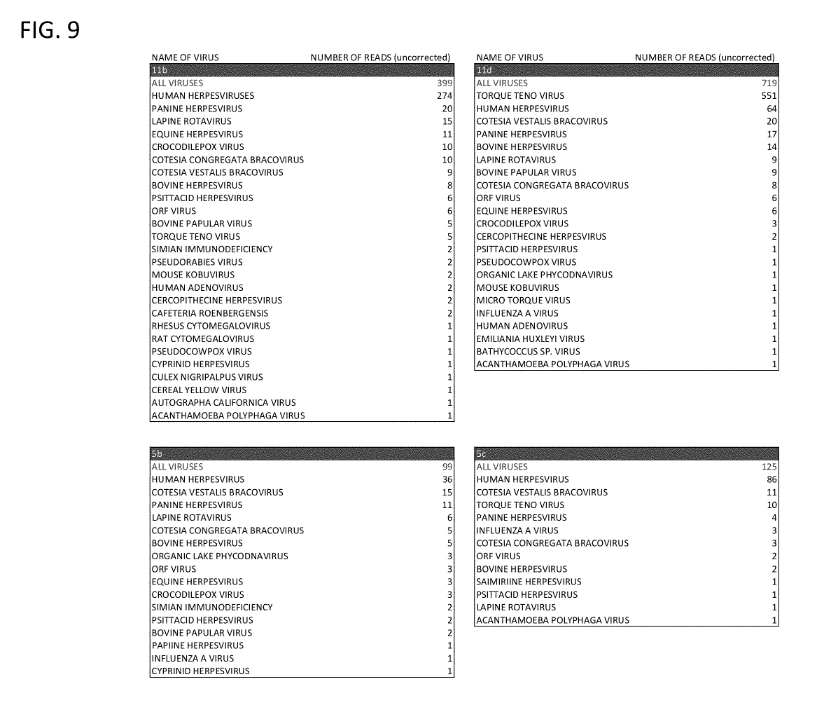

Samples 5b, 5c, 11b and 11d were carried through PathSeq analysis, as described in the main text of the manuscript. A detailed list of viral hits is indicated in FIG. 9.

### Example 4

### Identification of Bradyrhizobium enterica-Like Organisms

An environmental survey of patient care areas was carried out in order to establish a potential source of the infection. As the natural habitat of B. enterica was not known, the 16S ribosomal RNA sequence of the organism was used to query the NCBI nt (nucleotide) and wgs (whole genome sequence) databases. The “source” locations for the top 100 hits from each of the aforementioned homology searches were noted. Based on the results of this investigation, hospital-based water filtration systems were selected for testing. After PCR-based hospital environmental screening, various water sources from patient care areas were cultured on media that supports the growth of rhizobes. Briefly, 50 uL of each water source was plated on yeast mannitol agar (YMA) supplemented with either Congo Red (final concentration of 0.25 mg/mL) or bromothymol blue (BTB, final concentration of 0.25 mg/mL). Colonies of Bradyrhizobium species are described as excluding Congo red dye and thus maintaining a cream color and when grown on BTB, which is an acid-base indicator, secrete pH neutral to basic metabolites, thus keeping the BTB agar green to slightly blue in color.

Colonies that met the morphologic criteria expected for Bradyrhizobium species were streaked to isolation and were screened by PCR with Bradyrhizobium specific primers described above. A colony that grew after five days of incubation at 30° C., that was positive by this initial PCR. Genomic DNA from the organism was isolated and subjected to sequencing on a MiSeq platform (Illumina, San Diego, Calif.). The resulting reads were assembled into a genome of approximately 6.9 Mb in length using the AllPaths-LG software package. The draft genome that was assembled from this isolate represented an organism that was similar to, but not identical to B. enterica. This second novel organism was also determined to be in the genus Bradyrhizobium, based on a phylogenetic analysis (FIG. 10). It encoded a region of ˜152 kb that was identical to B. enterica. This region of the genome included all of the genes necessary for bacterial conjugation (transmission of genetic information, or “bacterial sex”, between different species of bacteria).

The identification of two novel bacteria within patient samples and the hospital in which they were cared for suggests that the hospital environment may be a source of many more novel organisms. As the conserved region in these two bacterial species encodes a “bacterial conjugation operon”, this region may be required, and is perhaps sufficient, for the evolution of novel organisms with pathogenicity to humans.

### Example 5

### Novel Approach for Identification of a Novel Viral, Prokaryotic or Eukaryotic Genome

The method used for the identification of a novel viral, prokaryotic or eukaryotic genome from sequencing data generated from a diseased tissue/body fluid specimen is described for the first time within this patent application.

This approach has been validated in the investigation of the gastrointestinal microbiome as demonstrated by the data presented herein, where a sequencing and computational method were employed for the successful identification of a new bacterium, Bradyrhizobium enterica, in a post-HSCT colitis syndrome.

Current microbiological methods used for diagnosis of human diseases in the clinical setting are biased to the identification of known organisms (with known growth, morphological, behavioral or sequence-based characteristics). Thus, the existing methods used bias against the discovery of unknown or unanticipated microorganisms. The method described by this work circumvents this inherent bias.

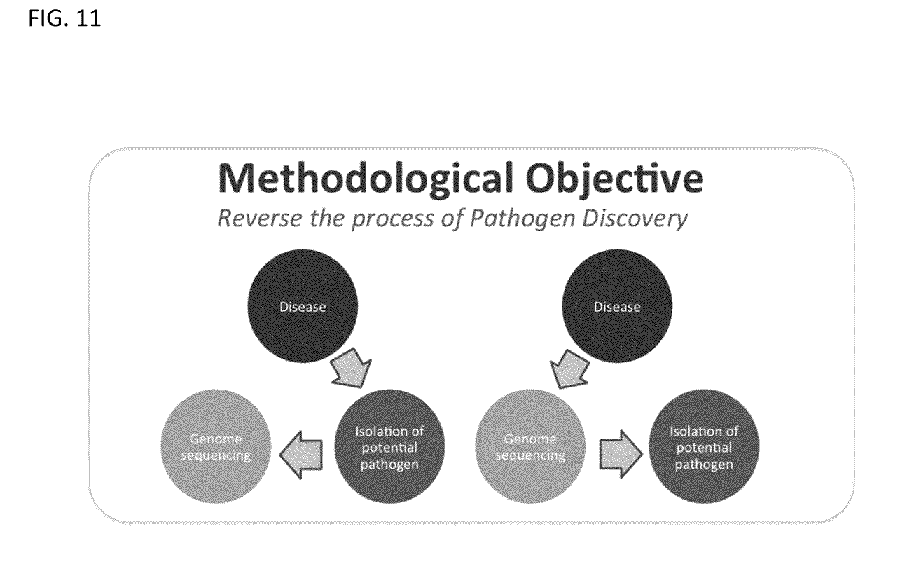

The methodological objective of “REVERSE MICROBIOLOGY”, the approach that has been demonstrated to be successful, is outlined in FIG. 11.

The first step of such an approach is to obtain diseased human or animal tissue or body fluid (or body secretion or excretion). Total DNA or RNA can be extracted from the sample (which is theorized to be a mixture of human and non-human microbial particles or cells as demonstrated in FIG. 12A.

The resultant DNA (or RNA) is subjected to next generation sequencing, which generates a mixed population of reads from human and other sources. These sequences may be quality filtered and are then taken forward for taxonomic classification (using a homology based classifier or alignment system; one possible approach is to use a program such as PathSeq (Kostic et al, Nature Biotechnology, 2011). Known microbial reads are assigned to a taxonomic classifier and the resultant data can be used for the identification of rare or abundant microorganisms that may be candidate pathogens. In most cases, a subset of reads will remain unclassifiable or “unmapped” (as outlined in FIG. 12B).

The remaining unmapped reads (or all nonhuman reads) can be taken forward for the generation of longer “contigs” or contiguous sequences that are generated by identifying regions of overlap between reads. This can be performed using computational methods that rely on “overlap consensus method”, de Bruijn graph theory based methods, or “greedy extension methods”. For the work described in the preliminary results section, we have used de Bruijn graph based assemblers in the programs VELVET and ALLPATHS. This results in the generation of longer sequences that are thought to comprise regions of the novel or divergent organism's genome (FIG. 12C).

Finally, the contigs are subjected to a host of tests carried out by a classifying program (such as GAEMR—www.broadinstitute.org/software/gaemr/) in order to determine which contigs likely belong to the same organism (as more than one organism without an existing draft genome may exist within the sample set) (FIG. 12D).

We claim:

1. An isolated bacterial strain comprising:
(i) at least one contiguous overlapping sequence (contig) selected from the group consisting of nucleic acid sequences of SEQ ID NOs: 1-88;
(ii) at least one contig selected from the group consisting of nucleic acid sequences of SEQ ID NOs: 94-349;
(iii) at least one open reading frame selected from the group consisting of nucleic acid sequences of SED ID Nos: 351-8212;
(iv) a bacterial conjugation operon of the SEQ ID NO: 350;
(v) a bacterium of ATCC Accession No. PTA-______1; or
(vi) a bacterium of ATCC Accession No. PTA-______2.

2. A pharmaceutical composition comprising a therapeutically effective amount of the bacterial strain of claim 1.

3. A vaccine comprising a therapeutically effective amount of attenuated or inactivated bacterial strain of claim 1.

4. A method of preventing, treating or alleviating a symptom of cord colitis syndrome in a subject comprising administering to the subject a therapeutically effective amount of a vaccine of claim 3.

5. A method of screening for an antibiotic agent against the bacterial strain of claim 1 comprising contacting a living bacterium with a candidate antibiotic agent and selecting an antibiotic agent that specifically inhibits growth of the bacterium.

6. A method for treating a bacterial infection in a subject comprising administering a therapeutically effective amount of an antibiotic agent screened according to the method of claim 5 to a subject suspect of or infected by the bacterial strain of claim 1.

7. A method of screening or monitoring water supply, water source, or a water filtration system comprising obtaining a sample from the water supply, water source, or water filtration system and detecting the presence of the bacterial strain of claim 1.

8. A method of identifying a novel viral, prokaryotic or eukaryotic genome, comprising
(i) collecting a nucleic acid sample from a biological sample obtained from a diseased subject;
(ii) performing a genome sequencing of the nucleic acid sample and generating a mix of reads;
(iii) identifying one or more unmapped reads; and
(iv) assembling the one or more unmapped reads into one or more contigs, thereby identifying a novel viral, prokaryotic or eukaryotic genome.

9. The method of claim 8, wherein the step of identifying one or more unmapped reads comprises taxonomic classification.

10. The method of claim 4, wherein the subject has a compromised immune system.

11. The method of claim 6, wherein the subject has a compromised immune system.
